(19) United States
(12) Patent Application Publication
(10) Pub. No.: US 2014/0271507 A1
(43) Pub. Date: Sep. 18, 2014

(54) Skin Compositions and Uses

(71) Applicant: Stemetrix, Inc., Woodland Hills, CA (US)

(72) Inventors: Dwain Morris-Irvin, Los Angeles, CA (US); Bradley Brownstein, Woodland Hills, CA (US)

(73) Assignee: Stemetrix, Inc., Woodland Hills, CA (US)

(21) Appl. No.: 14/210,328

(22) Filed: Mar. 13, 2014

Related U.S. Application Data
(60) Provisional application No. 61/778,428, filed on Mar. 13, 2013.

(51) Int. Cl. A61K 8/64; A61Q 19/00; A61K 8/35; A61K 8/49; A61Q 17/04; A61Q 15/00

(52) U.S. Cl. CPC A61K 8/64; A61Q 17/04; A61Q 15/00; A61K 8/35; A61K 8/4973; A61Q 19/00
USPC 424/59; 424/63; 424/65; 514/7.7

(57) ABSTRACT

The present invention provides a skin care composition for maintaining or improving the healthy appearance of the skin of an individual and/or rejuvenate the skin of an individual. A skin care composition comprises active ingredients, including, without limitation, growth factors, anti-oxidants, curcuminoids, oils and/or red rice extracts. Among the growth factors comprising biologically active ingredients of the skin care composition are insulin like growth factor (IGF), an epidermal growth factor protein (EGFP), an insulin-like growth factor (IGF), a transforming growth factor beta (TGFβ), and erythropoiesis stimulating agent (ESA). The invention further provides a skin care composition for topical use to maintain or improve the health of the skin of an individual and/or rejuvenate the skin of an individual.

This application is a U.S. Non-Provisional Patent Application that claims priority pursuant to 35 U.S.C. §119(e) to U.S. Provisional Patent Application 61/778,428, filed Mar. 13, 2013, the contents of which are incorporated by reference in its entirety.

### BACKGROUND

The invention relates to the preparation and use of a formulation for promoting healthy skin comprising one or more active ingredients

An individual's skin is under constant attack by environmental and other factors that can damage the skin. There are many lotions, creams, moisturizers and other products currently available that claim that they help to maintain or increase the health of an individual's skin. There are also many lotions, creams, moisturizers and other products healthy that claim that they help to rejuvenate an individual's skin. While many have some effect on the health of the skin, or may have an effect on rejuvenating an individual's skin, there is still a need for a product that is able to maintain or increase the health of an individual's skin and rejuvenate an individual's skin. The present invention provides a skin care composition that combines one or more of several active ingredients that provide a beneficial effect on the maintenance or improvement of the health of an individual's skin and/or is capable of a rejuvenation of an individual's skin through, at least in part, and without limitation, the stimulation of certain cell types associated with maintaining or increasing the health of the skin, while also rejuvenating the skin.

### SUMMARY

In an aspect of the present invention, a skin care composition comprises two or more active ingredients capable of maintaining or increasing the health of the skin of an individual and/or rejuvenating the skin of an individual and further, wherein the two or more active ingredients are selected, without limitation, from the group of growth factors, anti-oxidants, curcumins, oils and/or red rice extracts.

In an aspect of the present invention, a growth factor is selected from EGF, Heparin-binding EGF-like growth factor (HB-EGF), transforming growth factor-α (TGFα), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4), TGFβ1, TGFβ2, TGFβ3, inhibin-α, activin-β (forms A-C, E), anti-müllerian hormone, bone morphogenetic proteins (BMP1-11, & 15), and growth and differention factors (GDFs 1-3, 5-11) decapentaplegic, Lefty1, EPO, IGF1, IGF2, insulin and Nodal. In another aspect of the present invention, an anti-oxidant is selected from Vitamin C (ascorbate), vitamin B3 (niacinamide and its derivatives), Vitamin E (α-, β-, and γ-tocopherols, tocopherol sorbate, tocopherol acetate, other esters of tocopherol; phenols, such as butylated hydroxy benzoic acids and their salts, 6-hydroxy-2,5,7,8-tetramethylchroman-2-carboxylic acid (commercially available under the tradename TROLOX®); gallic acid and its alkyl esters, especially propyl gallate; uric acid and its salts and alkyl esters; sorbic acid and its salts; lipoic acid; amines (e.g., N,N-diethylhydroxylamine, amino-guanidine); sulfhydryl compounds (e.g., glutathione), dihydroxy fumaric acid and its salts; glycine pidolate; arginine pilolate; nordihydroguaiaretic acid; bioflavonoids; curcuminoids; lysine; methionine; proline; superoxide dismutase; silymarin; tea extracts; grape skin/seed extracts; melanin; and rosemary extracts.

In an aspect of the present invention, an active ingredient is a protein selected from EGF, epoetin (EPO), IGF1, and TGFβ3. In an aspect of the present invention, a sunscreen composition comprises a first and second biologically active ingredient wherein the first biologically active ingredient is an ESA and the second biologically active ingredient is selected from an EGFP, an IGF, and a TGFβ. In an aspect of the present invention, a skin care composition comprises a first biologically active ingredient is an ESA and the second biologically active ingredient is an EGFP. In an aspect of the present invention, a first biologically active ingredient is an ESA and a second biologically active ingredient is an IGF; or a first biologically active ingredient is an ESA and the second biologically active ingredient is a TGFβ; or a first biologically active ingredient is an ESA and a second biologically active ingredient is an IGF; or a first biologically active ingredient is an ESA and a second biologically active ingredient is a TGFβ.

In an aspect of the present invention, a first biologically active ingredient is an IGF and a second biologically active ingredient is a TGFβ. In an aspect of the present invention, a skin care composition comprises a first, a second, and a third biologically active ingredient, wherein the first biologically active ingredient is an ESA, the second biologically active ingredient is EGFP, and the third biologically active ingredient is an IGF; or comprises a first, a second, and a third biologically active ingredient, wherein the first biologically active ingredient is an ESA, the second biologically active ingredient is EGFP, and the third biologically active ingredient is a TGFβ; or comprises a first, a second, and a third biologically active ingredient, wherein the first biologically active ingredient is an ESA, the second biologically active ingredient is biologically active ingredient an IGF, and the third biologically active ingredient is a TGFβ; or comprises a first, a second, and a third biologically active ingredient, wherein the first biologically active ingredient is an EGFP, the second biologically active ingredient is an IGF, and the third biologically active ingredient is a TGFβ; or comprises a first, a second, a third and a fourth biologically active ingredient wherein the first biological active ingredient is that of an EGFP, the second biologically active ingredient is an IGF, and the third biologically active ingredient is a TGFβ, and fourth biologically active ingredient is an ESA.

In an aspect of the present invention, a skin care composition comprises, without limitation, an additional active ingredient selected from the group consisting of an anti-inflammatory, an anti-oxidant, and a humectant, including, without limitation, a curcuminoid and/or an ascorbate. In an aspect of the present invention, a skin care composition comprises, without limitation, a curcuminoid and an ascorbate; or a curcumin and ascorbic acid.

In an aspect of the present invention, the biological activity of the active ingredients is expressed in activity units, wherein each of the activities is present from between about 0.0001 U/ml and about 100 U/ml, between 0.001 U/ml and about 10 U/ml, between 0.01 U/ml and about 100 U/ml, between 0.1 U/ml and about 100 U/ml, between 1 U/ml and about 100 U/ml, between 0.01 U/ml and about 1000 U/ml, between 0.1 U/ml and about 500 U/ml, between 1 U/ml and about 100 U/ml and/or the protein is present at in the range of from about 0.01 U/ml to about 100 ng/ml, from about 0.1 pg to about 100 ng/ml, from about 1.0 pg/ml to about 400 ng/ml, from about 0.001 ng/ml to about 400 ng/ml, from about 0.01 ng/ml to about 400 ng/ml, from about 0.1 ng/ml to about 400 ng/ml, about 1.0 ng/ml to about 400 ng/ml, about 5 ng/ml to about 400 ng/ml, about 10 ng/ml to about 400 ng/ml, about 20 ng/ml to about 400 ng/ml, about 50 ng/ml to about 400 ng/ml, or about 100 ng/ml to about 1000 ng/ml and/or the biological activity is expressed as activity units (U) and the protein is present at in the range of from about 0.01 U/ml to about 10 U/ml, about 0.01 U/ml to about 15 U/ml, about 0.01 U/ml to about 20 U/ml, about 0.01 U/ml to about 25 U/ml, about 0.01 U/ml to about 30 U/ml, about 0.01 U/ml to about 35 U/ml, about 0.01 U/ml to about 40 U/ml, about 0.01 U/ml to about 45 U/ml, about 0.01 U/ml to about 50 U/ml, about 0.01 U/ml to about 75 U/ml, or about 0.01 U/ml to about 100 U/ml. In another aspect, the composition applied to a individual provides one or more proteins in an amount at least 0.01 U/ml, at least 0.01 U/ml/day, at least 0.1 U/ml, at least 1.0 U/ml, at least 5.0 U/ml, at least 10 U/ml, at least 20 U/ml, at least 50 U/ml, at least 100 U/ml, or at least 200 U/ml.

In an aspect of the present invention, a formulation comprises (a) two or more biologically active ingredient; and (b) a pharmaceutically or cosmetically acceptable carrier. In an aspect of the present invention, the carrier is the carrier between about 0.0001% (w/v) to about 99% (w/v), 0.0001% (w/v) to about 90% (w/v), 0.0001% (w/v) to about 80% (w/v), about 0.001% (w/v) to about 60.0% (w/v), or about 0.01% (w/v) to about 40.0% (w/v). In an aspect of the present invention, a carrier is in the form of a liquid, a gel suspension, ointment, cream, lotion, hydrogel, a paste; or a powder. In an aspect of the present invention, a carrier comprises water, petroleum jelly, petroleum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, and the like. In an aspect of the present invention the polymer is selected from a carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polyethylene glycol (PEG), branched PEG, POLYPEG®, polysialic acid (PSA), starch, hydroxyalkyl starch (HAS), hydroxylethyl starch (HES), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC).

In an aspect of the present invention, a carrier forms an emulsion selected from an oil-in-water emulsion and a water-in-oil emulsion. In an aspect of the present invention, a carrier forms a liposome. In an embodiment of the present invention, a formulation further comprises one or more of; a moisturizing agent or humectant, a pH adjusting agent, a deodorant or odor absorbing agent, a fragrance, a chelating agent, an emulsifing agent, a thickener, a solubilizing agent, a penetration enhancer, a colorant, a UV absorbent, an anti-oxidant agent, and a surfactant. In an embodiment of the present invention, a moisturizing or humectant agent is one or more of guanidine, glycolic acid and glycolate salts (e.g. ammonium salt and quaternary alkyl ammonium salt), aloe vera in any of its variety of forms (e.g., aloe vera gel), allantoin, argan oil (such as a preparation or extract of bark or seeds of the argan tree), urazole, polyhydroxy alcohols such as sorbitol, glycerol, hexanetriol, propylene glycol, butylene glycol, hexylene glycol and the like, polyethylene glycols, simple and complex saccharides and polysaccharides and derivatives (e.g., alkoxylated glucose), hyaluronic acid, lactamide monoethanolamine, acetamide monoethanolamine and any combination thereof.

In an aspect of the present invention, the pH of the pharmaceutical formulation has a pH value that ranges between about 3.0 and about 12.0, between about 5.0 and about 8.0, from about 4 to about 6, or from about 5 to about 7.5. In an aspect of the present invention, the solubilizing agent is one or more of citric acid, EDTA, sodium meta-phosphate, succinic acid, urea, cyclodextrin, polyvinylpyrrolidone, diethylammonium-ortho-benzoate, and micelle-forming solubilizers such as polysorbate, polyoxyethylene sorbitan, a fatty acid ester, polyoxyethylene n-alkyl ethers, n-alkyl amine n-oxides, polyoxamers, organic solvents such as acetone, phospholipids and cyclodextrins. In an embodiment of the present invention, the surfactant is one or more of a sarcosinate, glutamate, sodium alkyl sulfate, ammonium alkyl sulfate, sodium alkyl sulfate, ammonium alkyl sulfates, ammonium laureth-n-sulfate, sodium laureth-n-sulfate, an isothionate, glycerylether sulfonate, sulfosuccinate, sodium lauroyl sarcosinate, and monosodium lauroyl glutamate.

In an embodiment of the present invention, the biologically active protein of claim 3, wherein the protein is to be administered to a human or animal at amount of at least 0.01 ng/day, at least 0.01 ng/day, at least 0.1 ng/day, at least 1.0 ng/day, at least 5.0 ng/day, at least 10 ng/day, at least 20 ng/day, at least 50 ng/day, at least 100 ng/day, or at least 200 ng/day. In an aspect of the present invention, the formulation retains physical stability, retains chemical stability, and retains from 10 to 120% of the biological activity measured at the time of initial testing of the formulation. In an aspect of the present invention, a method for maintaining or improving the healthy appearance of the skin in an individual and or the rejuvenation of the skin of an individual that comprises topical administration to an individual of a skin care composition in an amount that maintains or improves the healthy appearance of the skin and/or rejuvenates the skin of an individual.

In an aspect of the present invention, the individual is suspected of having or has been diagnosed as having diabetes, celiac disease, acne (facial actinic keratoses), inflammatory conditions, has a decrease in circulating hormones such as growth hormone or estrogen, or the individual has been exposed to ultraviolet (UV) radiation. In an aspect of the present invention, a method of maintaining or improving or rejuvenating a skin feature or function in an individual by administering the skin care composition to an individual. In an aspect of the invention, the feature or function to be maintained or improved is selected from one or more of fine lines and wrinkles; age spots and dyspigmentation; decreased skin texture, tone and elasticity; roughness, photodamage; abnormal skin epidermal thickness; decreased skin thickness; decreased smoothness, tightness of skin; age spots; fine and coarse lines and wrinkles; fine and coarse periorbital wrinkles; deeper or more abundant nasolabial folds; facial fine and coarse lines; decreased skin radiance, decreased evenness of color or pigmentation; decreased skin firmness; hyperpigmentation; dark spots and/or patches; decreased skin brightness and healthy appearance; intrinsically and extrinsically aged skin; abnormal skin cellular turnover; decreased skin barrier; decreased skin hydration or ability to retain water; brown and red blotchiness; redness; reduction of dermal epidermal junction; loss of density or individual thickness of hairs; increased pore size and number of pores; or a combination thereof.

In an aspect of the present invention, the composition improves a feature of the skin by about 1% to about 100%, about 2% to about 98%, about 5% to about 90%, about 5% to about 80%, about 5% to about 70%, about 5% to about 60%, about 5% to about 40%, about 5% to about 30%, about 5% to about 20%, about 10% to about 80%, about 10% to about 70%, about 10% to about 60%, about 10% to about 50%, about 10% to about 40%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In an aspect of the present invention, the function improved is trans-epithelial water loss. In an aspect of the present invention, the feature improved is fine lines and wrinkles, redness, loss of density or individual thickness of hairs.

In an embodiment, a kit comprises a skin care composition comprising two or more active ingredients, wherein, one or more active ingredients are selected from the group of growth factors, anti-oxidants, curcumins, oils and/or red rice extracts for topical application in order to maintain or improve the healthy appearance of the skin of an individual and/or rejuvenate the skin of an individual.

### DETAILED DESCRIPTION

The present invention relates to a skin care composition comprising one or more active compounds, wherein the active ingredients when applied to the skin of an individual will help to maintain or increase the health of the skin and/or rejuvenate the skin. The skin care composition can include, but is not limited to, one or more active ingredients, and in another embodiment, includes, without limitation, two, three, four, five, six, seven or eight or more active compounds.

The practices described herein employ, unless otherwise indicated, conventional techniques of tissue culture, immunology, molecular biology, microbiology, cell biology and recombinant DNA, which are within the skill of the art. See, e.g., Harlow and Lane eds. (1999) Antibodies, A Laboratory Manual and Herzenberg et al. eds (1996) Weir's Handbook of Experimental Immunology.

All numerical designations, e.g., pH, temperature, time, concentration, and molecular weight, including ranges, are to be understood as approximations in accordance with common practice in the art. When used herein, the term “about” may connote variation (+) or (−) 1%, 2%, 3%, 4%, 5%, 6%, 7%, 8%, 9%, or 10% of the stated amount, as appropriate given the context. It is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a pharmaceutically acceptable carrier” includes a plurality of pharmaceutically acceptable carriers, including mixtures thereof. On the other hand “one” designates the singular.

As used herein, the term “comprising” is intended to mean that the compositions and methods include the listed elements, but do not exclude other unlisted elements. “Consisting essentially of” when used to define compositions and methods, excludes other elements that alters the basic nature of the composition and/or method, but does not exclude other unlisted elements. Thus, a composition consisting essentially of the elements as defined herein would not exclude trace amounts of elements, such as contaminants from any isolation and purification methods or pharmaceutically acceptable carriers, such as phosphate buffered saline, preservatives, and the like, but would exclude additional unspecified amino acids. “Consisting of” excludes more than trace elements of other ingredients and substantial method steps for administering the compositions described herein. Embodiments defined by each of these transition terms are within the scope of this disclosure and the inventions embodied therein.

As used herein, the term “formulation(s)” means a combination of at least one active ingredient with one or more other ingredient, also commonly referred to as excipients, which may be independently active or inactive. The term “formulation”, may or may not refer to a pharmaceutically acceptable composition for administration to humans or animals, and may include compositions that are useful intermediates for storage or research purposes. In an embodiment, administration to humans or animals may include, without limitation, topical, sublingual, rectal, vaginal, transcutaneous, oral, inhaled, intranasal, pulmonary, subcutaneous, intravenous, enteral or parenteral.

As used herein, a protein can be a plasma derived protein or a recombinant protein. Production of a plasma derived protein can be through methods known in the art, including those related to the fractionation of blood plasma, colostrum or milk. Production of a recombinant therapeutic protein includes any method known in the art for (i) the production of recombinant DNA by genetic engineering, (ii) introducing recombinant DNA into prokaryotic or eukaryotic cells by, for example and without limitation, transfection, electroporation or microinjection, (iii) cultivating said transformed cells, (iv) expressing therapeutic protein, e.g. constitutively or upon induction, and (v) isolating said recombinant protein, e.g. from the culture medium or by harvesting the transformed cells, in order to obtain purified therapeutic protein. In other aspects, the therapeutic protein is produced by expression in a suitable prokaryotic or eukaryotic host system characterized by producing a pharmacologically acceptable blood coagulation protein molecule. Examples, without limitation, of eukaryotic cells are mammalian cells, such as CHO, COS, HEK 293, BHK, SK-Hep, and HepG2. A wide variety of vectors are used for the preparation of the therapeutic protein and are selected from eukaryotic and prokaryotic expression vectors. Examples, without limitation, of vectors for prokaryotic expression include plasmids such as, and without limitation, pRSET, pET, and pBAD, wherein the promoters used in prokaryotic expression vectors include one or more of, and without limitation, lac, trc, trp, recA, or araBAD. Examples, without limitation, of vectors for eukaryotic expression include: (i) for example, without limitation, for expression in yeast, vectors such as, and without limitation, pAO, pPIC, pYES, or pMET, using promoters such as, and without limitation, AOX1, GAP, GAL1, or AUG1; (ii) for example, without limitation, for expression in insect cells, vectors such as and without limitation, pMT, pAc5, plB, pMIB, or pBAC, using promoters such as and without limitation PH, p10, MT, Ac5, OplE2, gp64, or polh, and (iii) for example, without limitation, for expression in mammalian cells, vectors such as and without limitation pSVL, pCMV, pRc/RSV, pcDNA3, or pBPV, and vectors derived from, in one aspect, viral systems such as and without limitation vaccinia virus, adeno-associated viruses, herpes viruses, or retroviruses, using promoters such as and without limitation CMV, SV40, EF-1, UbC, RSV, ADV, BPV, and β-actin.

In order that the present invention may be more readily understood, certain abbreviations and terms are first defined. Additional definitions are set forth throughout the detailed description.

All publications, including but not limited to patents and patent applications, cited in this specification are herein incorporated by reference as though fully set forth.

In an embodiment, rejuvenation or maintains or improves the health of the skin, including without limitation, the maintenance or improvement in the appearance of the skin is meant a maintenance and/or a restoration of the appearance of healthy skin and a decrease in the visible signs of damage to the skin of an individual, including, without limitation, aging or skin damage, redness, loss of radiance or decrease in light reflectivity, discoloration, enlarged pores, uneven skin tone, coarse or fine wrinkles, dryness, loss of firmness, loss of smoothness, and uneven pigmentation.

In an embodiment, skin” or epithelium” is meant an outer cutaneous membrane of a human or non-human animal, comprising of an epidermis and the underlying dermis. It will be understood that the skin is an epithelial membrane comprised of an epidermal layer (the epidermis) and a dermal layer (the dermis). The epidermis includes a layer of germinating cells, the basal layer or stratum germinativum, which undergo mitotic division producing cells of the epidermal layer. In addition to acting as a protective barrier, the skin harbors specialized immune cells or mobile macrophages and allows for migration other cells participating in immune surveillance or responses. While the epidermis consists of a protective, water resistant barrier of stratified and nonvascularized cells, the dermis comprises a highly vascularized and innervated tissue layer. Accessory structures in the dermis include; hair, nails, and a variety of multicellular exocrine glands, such as sweat glands.

The term “protein” is used in its broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. A peptide of three or more amino acids is commonly called an oligopeptide if the peptide chain is short. If the peptide chain is long, the peptide is commonly called a polypeptide or a protein.

In an embodiment, a stem cell refers to a germinal cell and/or a multi- or pluripotent cell meaning that the cells are capable of dividing to produce daughter cells and daughter cells thereof are capable of differentiating to exhibit morphology or functions different from the parent stem cell.

In an embodiment, “EGF” is human epidermal growth factor which is transcribed as 1207 amino acid prepropeptide (UniProt, EGF_HUMAN) and processed to the 53 amino acid mature EGF (residues 971-1023 of P01133) having 3 internal disulfide linkages. EGF has been called urogastrone (URG) and is also known as HOMG4, pro-epidermal growth factor. In an embodiment, EGF is a derivative, a truncated form, a mimetic, an aptamer, a mutated form, or other non-naturally occurring form of EGF.

In an embodiment, “erythropoietin”, “EPO” or “rhEPO” is meant a composition which is a polypeptide chain monomer synthesized in the human body, by a human cell induced to express an endogenous gene, or made recombinantly having a 165-166 amino acids (Uniprot accession No. P01588 mature chain) and active truncations thereof capable of activating the erythropoietin receptor. EPO and rhEPO are generally glycoproteins of 30 to 34 kDa. rhEPO is also known as a component of several pharmaceutical products given the nonproprietary name, epoietin. For example, epoetin alpha, is known by brand names EPOGEN®, EPREX®, and PROCRIT®. Reference to “rhEPO” may include active cleavage products, especially C-terminal truncations, be glycosylated or non-glycosylated, or be otherwise modified such through linkage to a water soluble polymer, including, without limitation those polymers attached by PEGylation, PSAylation, HESylation or carbamoylation at specific or non-specific sites on the polypeptide chain. In an embodiment, EPO is a derivative, a truncated form, a mutated form, mimetic, aptamer, or other non-naturally occurring form of EPO.

In an embodiment, “ESA” is an erythropoiesis stimulating agent that can activate the EPOR to stimulate erythroblast proliferation and differentiation and includes EPO as well as modified forms of the erythropoietin. Some ESA are EPO-derived or EPO biosimilars, meaning that they have substantial sequence identity to erythropoietin. By substantial sequence identity is meant that, using a sequence alignment algorithm, the sequence of the EPO-derived ESA and erythropoietin can be matched and the percent identity between the two sequences is greater than 80%. Examples of erythropoietin-derived products include darbepoetin alfa (ARANESP™, Amgen, Calif.) which comprises a variant polypeptide chain sequence of rhEPO as described in U.S. Pat. No. 7,217,689 and C.E.R.A. (Continuous erythropoiesis receptor activator) also known by its chemical name, methoxypolyethylene glycol-epoetin beta, and methoxy polyethylene glycol-epoetin beta (MICERA, Roche, Switzerland), and others (EP1196443B1). Non-erythropoietin derived ESAs include erythropoietin mimetic peptides (EMP), such as EMP-1 (Affymax), and other synthetic peptides such as peginesatide OMONTYS® (Affymas and Takeda). By “EPO-mimetic peptide” is meant a composition having natural, or a combination of natural and non-natural amino acid residues connected in sequence whereby substantially none of the sequence can be aligned with naturally occurring erythropoietin but where the erythropoietin-mimetic peptide exhibits erythropoietic activity which is similar to erythropoietin, such as but not limited to EPO-R specific binding and stimulation of UT7 cell proliferation. In an embodiment, ESA is a derivative, a truncated form, a mutated form, mimetic, aptamer, or other non-naturally occurring form of ESA.

In an embodiment, “IGF1” is insulin-like growth factor-1 (UniProt P05019 (IGF1_HUMAN isoforms 1-3)) is processed to the mature 70 amino acid basic protein representing residues 49-118 of P05019, having three internal disulfide bonds with a molecular weight of 7,649 Da. IGF1 is also known formerly as somatomedin C, and IBP1, IGF-1, IGF1A, IGF1B, IGFI, IGF-I, Insulin-like growth factor I, Mechano growth factor, MGF, Somatomedin-C. By “IGF2” is meant insulin-like growth factor 2 (UniProt P01344) also known as Cell growth-inhibiting gene 44 protein, IGF-II, Insulin-like growth factor II, Insulin-like growth factor II-associated protein, and Somatomedin-A, a 67 amino acid neutral polypeptide. In an embodiment, IGF-1 or IGF-2 is a derivative, a truncated form, a mutated form, mimetic, aptamer, or other non-naturally occurring form of IGF-1 or IGF-2.

In an embodiment, “TGFβ3” is the human protein transforming growth factor beta-3 (UniProt P10600) a 412 preproprotein which is processed to an active molecule of 112 amino acids (24 kDa) having four internal disulfide cross-links and, potentially, one intermolecular disulfide. TGF-β3 has also been known as arrhythmogenic right ventricular dysplasia 1, ARVD, ARVD1, FLJ16571, TGF-β3, TGF-beta-3, Transforming growth factor beta-3. In an embodiment, TGFβ3 is a derivative, a truncated form, a mutated form, mimetic, aptamer, or other non-naturally occurring form of TGFβ3.

In an embodiment, “curcumin” is the specific compound 1E,6E)-1,7-Bis(4-hydroxy-3-methoxyphenyl)-1,6-heptadiene-3,5-dione, CAS 458-37-7 or a preparation containing substantially pure curcumin. In an embodiment, curcumin is a derivative, a truncated form, a mutated form, mimetic, aptamer, or other non-naturally occurring form of curcumin.

In an embodiment, “Vitamin C”, “Vit C”, or “ascorbic acid” refers to L-hexuronic acid, (5R)-[(1S)-1,2-dihydroxyethyl]-3,4-dihydroxyfuran-2(5H)-one, CAS Reg. No. 50-81-7 or a preparation containing substantially pure ascorbic acid. In an embodiment, Vitamin C is a derivative, a truncated form, a mutated form, mimetic, aptamer, or other non-naturally occurring form of Vitamin C.

In an embodiment, Argan oil is a plant oil produced from the kernels of the argan tree. In an embodiment, Argan oil contains tocopherols (vitamin E), phenols, carotenes, squalene, and fatty acids, (80% unsaturated fatty acids). The main natural phenols in argan oil are caffeic acid, oleuropein, vanillic acid, tyrosol, catechol, resorcinol, (−)-epicatechin and (+)-catechin.

In an embodiment, red rice, is a species of rice (Oryza) and has been described as containing high levels of anti-oxidants.

In an embodiment, the term “individual” includes any human, nonhuman primate, or nonhuman animal, a eukaryotic cell, a tissue culture, or a tissue of an animal, e.g. a mammal, including a human. Non-human animals individual to treatment include, for example, without limitation, a simian, a murine animal, a canine, a leporid, such as a rabbit, livestock, sport animals, and companion animals. In a further embodiment, a non-human animal is a reptile, a bird, a marsupial or other animal. In an additional embodiment, a nonhuman animal is a cat, a dog, a cow, a horse, a goat, a sheep, a pig or other domestic or non-domesticated animal.

In an embodiment, the term “recombinant protein may include any recombinant protein, heterologous or naturally occurring, obtained via recombinant DNA technology, or a biologically active derivative thereof. In certain embodiments, the term encompasses proteins as described above and nucleic acids, encoding a recombinant protein of the invention. Such nucleic acids include, for example and without limitation, genes, pre-mRNAs, mRNAs, polymorphic variants, alleles, synthetic and naturally-occurring mutants. Recombinant engineering techniques, are described in U.S. Pat. No. 4,757,006; U.S. Pat. No. 5,733,873; U.S. Pat. No. 5,198,349; U.S. Pat. No. 5,250,421; U.S. Pat. No. 5,919,766; EP 306 968.

Proteins comprising polypeptides longer than about 50 to 100 amino acids in length or commonly made by recombinant methods well known in the art. Glycoproteins are typically produced in eukaryotic host cells having capable of decorating the proteins at specified amino acid motifs with polysaccharide structures also called glycans. The activity of the glycoprotein may be altered by the presence or absence of the glycan. The polypeptide growth factors of the invention are available from commercial sources or can be produced and purified by methods that are well known in the art. In an embodiment the one or more of an EGFP, an ESA, an IGF, and a TGFβ protein is available as an aqueous solution. In another aspect one or more of EGFP, an ESA, an IGF, and a TGFβ protein is available as a lyophilized powder.

In an embodiment a protein is a naturally derived protein. In a further embodiment, a protein is a recombinant protein. In an additional embodiment, a protein, either naturally occurring or recombinant, is a full-length protein, precursors of the protein, biologically active or functional subunits or fragments of the protein, and functional derivatives thereof, as well as variants thereof.

In an embodiment, “endogenous protein” includes a protein which originates from the mammal intended to receive treatment. The term also includes a protein transcribed from a transgene or any other foreign DNA present in said mammal. As used herein, “exogenous protein” includes a protein which does not originate from said mammal.

In an embodiment, a variant (or analog) polypeptides include insertion variants, wherein one or more amino acid residues are added to a protein amino acid sequence of the invention. Insertions may be located at either or both termini of the protein, and/or may be positioned within internal regions of the protein amino acid sequence. Insertion variants, with additional residues at either or both termini, include for example, fusion proteins and proteins including amino acid tags or other amino acid labels. In one aspect, a protein molecule may optionally contain an N-terminal Met, especially when the molecule is expressed recombinantly in a bacterial cell such as E. coli.

In an embodiment, a deletion variant includes, without limitation, one or more amino acid residues in a protein as described herein are removed. Deletions can be effected at one or both termini of the protein, and/or with removal of one or more residues within the protein amino acid sequence. Deletion variants, therefore, include all fragments of a protein polypeptide sequence.

In an embodiment, a substitution variant includes, without limitation, one or more amino acid residues of a protein are removed and replaced with alternative residues. In one aspect, the substitutions are conservative in nature and conservative substitutions of this type are well known in the art. Alternatively, the invention embraces substitutions that are also non-conservative. Exemplary conservative substitutions are described in Lehninger, [Biochemistry, 2nd Edition; Worth Publishers, Inc., New York (1975), pp. 71-77] and set out immediately below.

Loss of the integrity and loss of appearance of the skin are interconnected by the loss of structural elements of the cells and tissues of the skin. Compromise of skin appearance and/or function occurs during the natural aging process and due to certain pathological conditions or natural elements such as diabetes, celiac disease, acne (facial actinic keratoses), inflammatory conditions, by the decrease in circulating hormones such as growth hormone or estrogen, exposure to the ultraviolet (UV) radiation from the sun, by a dietary deficiency, by excessive intake of alcohol, smoking, and the effects of gravity. Loss of integrity of the components of the skin may include one of more of: a decline in the germinative cell activity, a decrease in the thickness of the epidermis, reduction in number or migration of cells, reduction in number or migration of immune cells, decrease in exocrine or glandular activity, loss of number or function of the vessels, loss of germinative capacity of the hair follicles, and loss in extracellular matrix and/or collagen production or collagen maturation. Reversal of these effects would be beneficial.

The present invention is based on the use of compounds including growth factors known to promote wound healing and, in particular, to promote cell proliferation and activity, including, without limitation, the stimulation of stem cells. Renewal or rejuvenation and healthy appearance of the skin is dependent upon, without limitation, such cellular functions as proliferation, differentiation, migration, and protein synthesis to maintain or regenerate the cells, tissues, matrix, and accessory structure and their proper functions. In certain embodiments, the formulation of the present invention improves, without limitation, the functions of or healthy appearance of skin or rejuvenates the skin in one or more aspects. In aspects of the invention, the application of the formulation to a individual rejuvenates the skin by affecting one or more of the functions or features of the skin including, without limitation, fine lines and wrinkles; age spots and dyspigmentation; decreased skin texture, tone and elasticity; roughness, photodamage; abnormal skin epidermal thickness; decreased skin thickness; decreased smoothness, tightness of skin; age spots; fine and coarse lines and wrinkles; fine and coarse periorbital wrinkles; deeper or more abundant nasolabial folds; facial fine and coarse lines; decreased skin radiance, decreased evenness of color or pigmentation; decreased skin firmness; hyperpigmentation; dark spots and/or patches; decreased skin brightness and healthy appearance; intrinsically and extrinsically aged skin; abnormal skin cellular turnover; decreased skin barrier; decreased skin hydration or ability to retain water; brown and red blotchiness; redness; reduction of dermal epidermal junction; loss of density or individual thickness of hair; increased pore size and number of pores; or a combination thereof.

In an embodiment, a skin care composition comprises a formulation to maintain the integrity of skin stem cells and promote their function in an animal, in need thereof, including promoting endothelial cell growth and microvessel integrity. In an embodiment, a skin care composition comprises a combination of one or two or more active ingredients. In an embodiment, an active ingredient is, without limitation, a protein, a vitamin, a small molecule or other molecule that affects the maintenance or increase in health of skin and/or rejuvenates skin. In an embodiment, a protein is, without limitation, a growth factor. In another embodiment, an active ingredient is, without limitation, an ascorbate compound, a curcuminoid, an oil, an extract of red rice and, optionally, other active ingredients representing compounds that may have one or more effects on the skin wherein the effect promotes the maintenance or increase in the healthy appearance of the skin. In an embodiment, the skin care composition includes, without limitation, an anti-oxidant, an anti-inflammatory agent, and a humectant.

Whereas, many growth factors have been identified, they have been classified into “families” based on their structural domains and receptor binding properties as shown in Table 1.

TABLE 1

| Growth Factor | |
|---|---|
| Family Type | Additional Family Members |
| EGF | EGF, Heparin-binding EGF-like growth factor (HB- |
| Protein | EGF), transforming growth factor-α (TGFα), |
| (EGFP) | Amphiregulin (AR), Epiregulin (EPR), Epigen, |
| | Betacellulin (BTC), neuregulin-1 (NRG1), neuregulin-2 |
| | (NRG2), neuregulin-3 (NRG3), neuregulin-4 (NRG4) |
| ESA | Native or recombinant erythropoietin (EPO or rhEPO). |
| IGF | IGF1, IGF2, insulin |
| TGFβ | TGFβ, TGFβ2, TGFβ3, inhibin-α, activin-β (forms A-C, |
| | E), anti-müllerian hormone, bone morphogenetic proteins |
| | (BMP1-11, & 15), and growth and differention factors |
| | (GDFs 1-3, 5-11) |
| | decapentaplegic, Lefty1, and Nodal. |

In an embodiment, “an EGFP” includes, without limitation, a protein having an EGF-like domain according to Table 1.

In an embodiment, a skin care composition includes, without limitation, an ESA. In a further embodiment, a skin care composition includes, without limitation, a protein, synthetic construct, or peptide capable of binding an EPOR or capable of promoting non-erythropoietic functions of EPO or rhEPO (epoetin), and includes, without limitation EPO-derived analogs and non-EPO analogs or peptides that activate the EPOR with varying affinities.

In an embodiment, a skin care composition includes, without limitation, GM-CSF, IL3, and/or IL5

In an embodiment, a skin care composition includes, without limitation, an IGF. In a further embodiment, a skin care composition includes, without limitation, a polypeptide structurally related to insulin and capable of binding the IGF receptor (IGFR), the insulin receptor (ICD220, HHF5, Insulin receptor, IR) and/or a. recombinant form of IGF1 and complexes thereof, mecasermin (brand name INCRELEX®) a synthetic analog of IGF-1. In an embodiment, a skin care composition includes, without limitation, IPLEX® (Insmed), mecasermin rinfabate, which is the human recombinant form of the naturally occurring protein complex of insulin-like growth factor-I (IGF-1) and insulin-like growth factor binding protein-3 (IGFBP-3).

In an embodiment, “a TGF-β” is a structurally and functionally related member of the TGF-β family of proteins (Table1), and the TGF-β family includes several bone morphogenic proteins (BMPs) and growth and differentiation factor (GDF) proteins. GDF5 (UniProt P43026, BMP14, Cartilage-derived morphogenetic protein 1, CDMP1).

In an embodiment, a curcuminoid includes, but is not limited, curcumin, tetrahydrocurcumin, desmethoxycurcumin and bis-desmethoxycurcumin and their esters, alone or in combination, naturally present in turmeric, a spice prepared from the rhizomes (underground stems) of the plant Curcuma longa.

In an embodiment, ascorbate includes, without limitation, ascorbic acid and its salts and various oxidation states including, ascorbyl esters of fatty acids, (e.g., ascorbic acid 2-phosphate, magnesium ascorbyl phosphate, sodium ascorbyl phosphate, retinyl ascorbate, ascorbyl palmitate, ascorbyl sorbate, and tetrahexyldecyl ascorbate) ascorbic acid derivatives and dehydroascorbic acid (DHA). Ascorbate is an antioxidant

In an embodiment the invention comprises, without limitation, a skin care composition that is formulated for application to the skin of an individual and comprises, without limitation, one or more of: an EGFP, an ESA, an IGF and/or a TGFβ. In another embodiment, the skin care composition includes, without limitation, one or more additional compatible active ingredients which provide the composition with a pharmaceutical, cosmeceutical or cosmetic effect, and this skin care composition can include, without limitation, one, two or more an EGFP, an ESA, an IGF or a TGFβ.

In an embodiment, the skin care composition comprises, without limitation, one of an EGFP, an ESA, an IGF, or a TGFβ and a pharmaceutically, cosmeceutically or cosmetically active ingredient. In another embodiment the skin care composition comprises, without limitation, one or more of an EGFP, an ESA, an IGF, and a TGFβ and a curcuminoid. In another embodiment the skin care composition comprises, without limitation, one or more of an EGFP, an ESA, an IGF, and a TGFβ and an ascorbate. In a further embodiment, the skin care composition comprises, without limitation, an EGFP, an ESA, an IGF, and a TGFβ, and an ascorbate and a curcuminoid.

In an embodiment, the skin care composition comprises, without limitation, two of an EGFP, an ESA, an IGF, and a TGFβ. In an embodiment, the skin care composition comprises two members of the growth factor families selected, without limitation, from an EGFP and an ESA, EGFP and an IGF, an EGFP and a TGFβ, an ESA and an IGF, and ESA and a TGFβ, and an IGF and TGFβ.

In an embodiment, the skin care composition comprises, without limitation, three of an EGFP, an ESA, an IGF, and a TGFβ. In aspect further embodiment, the composition comprises, without limitation, an EGFP, an ESA, and an IGF. In an embodiment, the skin care composition comprises, without limitation, an EGFP, an ESA, and a TGFβ. In a further embodiment, the skin care composition comprises, without limitation, an EGFP, IGF, and a TGFβ. In an aspect the skin care composition comprises an ESA, an IGF, and a TGFβ. In another embodiment, the skin care composition comprises, without limitation, an EGFP, an ESA, an IGF, and a TGFβ. In another embodiment, the skin care composition comprises, without limitation, an EGF, an IGF, a TGFβ3, and an Erythropoietin-α.

In another embodiment the skin care composition comprises, without limitation, an EGFP, an ESA, an IGF, a TGFβ, and an ascorbate. In another embodiment the skin care composition comprises, without limitation, an EGFP, an ESA, an IGF, a TGFβ and a curcuminoid. In another embodiment the skin care composition comprises, without limitation, an EGFP, an ESA, an IGF, a TGFβ, an ascorbate, and a curcuminoid. In a further embodiment, additional pharmaceutically effective, cosmeceutically effective or cosmetically effective compounds may be added to any of the aforementioned skin care compositions such that the formulation has a desired effect or an additional beneficial effect, including, without limitation, maintenance or increase in the health of the skin and/or skin rejuvenation.

In an embodiment, a skin care composition comprises one or more active ingredients. In a further embodiment, a skin care composition comprises one or more active ingredients selected from, without limitation, growth factors, ascorbate, a vitamin, a curcuminoid, argan oil and/or an extract of red rice. In an embodiment, a growth factor includes, without limitation, an EGFP, a TGFβ, and ESA and or an IGF.

Other than a the combination of active compounds providing the desired effect of maintaining or improving the health of skin or rejuvenating the skin in one or more aspects such as enhancing the integrity and activity of cells, tissues, and accessory structures of the skin; a skin care composition comprises, without limitation an additional compound or ingredient that exerts a pharmacological, cosmeceutical, or cosmetic or any other beneficial activity including, without limitation, an agent that prevents or reduces pain, itching, roughness, or inflammation. An “other beneficial activity” is one that can, but is not limited to, a beneficial activity that is only perceived as such by the individual using the inventive compositions.

In an embodiment, an additional active ingredient is, without limitation, an extract of a herbaceous plant or portion thereof, such as ginseng root, or an additional Vitamin, such as retinol (Vitamin A) or tocopherol (Vitamin E), a steroidal and non-steroidal anti-inflammatory agent, or other materials such as aloe vera, chamomile, alpha-bisabolol, cola nitida extract, green tea extract, tea tree oil, licorice extract, allantoin, caffeine or other xanthines, glycyrrhizic acid and their derivatives.

In an embodiment, a skin care composition of the present invention includes, without limitation, individually or in combination; Argan oil (oil derived from the Argan tree or seeds) and a component of red (Himalayan) rice, Ozonized Oryza Sativa Callus Culture Extract Red Rice (REGENISTEM™, Lonza), as additional active ingredients.

In an embodiment, a method of preparing a pharmaceutical or cosmetic or a pharmaceutically active topical preparation (a “cosmeceutical”) that improves the function of or appearance of skin, the maintenance or improvement in skin, including the appearance of the skin, and/or rejuvenation of the skin of an individual is disclosed herein. In an embodiment, a method disclosed herein comprises, without limitation, the step of contacting an active ingredient as disclosed herein, including, without limitation, one or more of an EGFP, an ESA, an IGF, and a TGFβ; and/or a curmunoid, and/or an ascorbate; with one or more pharmaceutically or cosmetically acceptable ingredients as disclosed herein to prepare a formulation. The contacting step may be repeated for each and every active component and, optionally, in the presence or absence of other active or non-active ingredients. The formulation so formed may be combined with a second, a third, a fourth, a fifth, a six, a seventh, an eighth, and a ninth or more formulation as an intermediate or a final step to form the an intermediate or final formulation. A formulation prepared using one or more contacting steps as described may be used in the method of the invention to rejuvenate or improve the appearance or function of the skin.

In an embodiment, the skin care composition of the invention is supplied as a kit wherein one or more of the actives selected from an EGFP, an ESA, an IGF, and a TGFβ, a curmunoid, and/or an ascorbate, argan oil, or a derivative of red rice is present in a container, including, without limitation an ampoule, a vial, a tube, a bottle, a packet, or a syringe in the kit. In an embodiment, a bottle includes, without limitation, a squeeze bottle, a bottle with a pump mechanism that when depressed provides a skin care composition to an individual, a pour bottle or other type of bottle. In a further embodiment, a kit contains a diluent and/or carrier supplied in the same container as the active ingredient or active ingredients or in a separate container. In an embodiment, a kit comprises instructions for the preparation of the formulation by contacting the contents of the ampoule, vial, tube, bottle, packet, or syringe with the diluent or carrier.

In an embodiment of the invention, a mixture comprising one or more of an EGFP, an ESA, an IGF, and a TGFβ protein is produced by culturing cells under conditions where the cells secrete the polypeptides into the growth medium and the medium is used in the formulation of a skin care composition to promote the maintenance or improvement in the skin and/or rejuvenation of the skin, including without limitation, maintaining or improving the appearance of skin.

In an embodiment, the proteins or extracts used as either an active or inactive ingredient of a skin care composition includes, without limitation, compounds which are obtained from natural sources, including, without limitation, plant sources.

In an embodiment, the presence and quantity of specific proteins, such as members of the growth factor protein families designated herein as an EGFP, an ESA, an IGF, and a TGFβ protein present in a skin care composition, are determined by a solid phase assay such as an immunocapture assay, including, without limitation, an ELISA or RIA regardless of the final detection or read-out method. In a further embodiment, additional methods of quantitating specific substances in complex mixtures include, without limitation, an immunocapture or ligand binding method. In a further embodiment, the presence of a biological activity of a protein and other active ingredients, such as curcumoinds and ascorbate, is determined by measuring a biological response to a liquid or other composition or formulation and comparing the activity to a standard curve using known amounts or units of biological activity.

In an additional embodiment, an active ingredient, including a protein, which includes, but is not limited to a growth factor, contained in a skin care composition that has enzymatic or other measurable activity should comprise from about 0.01 U/ml to about 10 U/ml, about 0.01 U/ml to about 15 U/ml, about 0.01 U/ml to about 20 U/ml, about 0.01 U/ml to about 25 U/ml, about 0.01 U/ml to about 30 U/ml, about 0.01 U/ml to about 35 U/ml, about 0.01 U/ml to about 40 U/ml, about 0.01 U/ml to about 45 U/ml, about 0.01 U/ml to about 50 U/ml, about 0.01 U/ml to about 75 U/ml, or about 0.01 U/ml to about 100 U/ml of the skin care composition or at least 0.01 U/ml, 0.05 U/ml, 0.1 U/ml, 0.15 U/ml, 0.2 U/ml, 0.25 U/ml, 0.3 U/ml, 0.4 U/ml, 0.5 U/ml, 0.6 U/ml, 0.7 U/ml, 0.8 U/ml, 0.9 U/ml, 1 U/ml, 1.25 U/ml, 1.5 U/ml, 1.75 U/ml, 2 U/ml, 2.25 U/ml, 2.5 U/ml, 2.75 U/ml, 3 U/ml, 3.25 U/ml, 3.5 U/ml, 3.75 U/ml, 4 U/ml, U/ml, 4.25 U/ml, 4.5 U/ml, 4.75 U/ml, 5 U/ml, 5.25 U/ml, 5.5 U/ml, 5.75 U/ml, 6 U/ml, 6.25 U/ml, 6.5 U/ml, 6.75 U/ml, 7 U/ml, 7.25 U/ml, 7.5 U/ml, 7.75 U/ml, 8 U/ml, 8.5 U/ml, 9 U/ml, 9.5 U/ml, 10 U/ml, 10.5 U/ml, 11 U/ml, 11.5 U/ml, 12 U/ml, 12.5 U/ml, 13 U/ml, 13.5 U/ml, 14 U/ml, 14.5 U/ml, 15 U/ml, 15.5 U/ml, 16 U/ml, 16.5 U/ml, 17 U/ml, 17.5 U/ml, 18 U/ml, 18.5 U/ml, 19 U/ml, 19.5 U/ml, 20 U/ml, 21 U/ml, 22 U/ml, 23 U/ml, 24 U/ml, 25 U/ml, 30 U/ml, 35 U/ml, 40 U/ml, 45 U/ml, 50 U/ml, 55 U/ml, 60 U/ml, 65 U/ml, 70 U/ml, 75 U/ml, 80 U/ml, 85 U/ml, 90 U/ml, 95 U/ml, 100 U/ml, 110 U/ml, 120 U/ml, 125 U/ml, 130 U/ml, 140 U/ml, 150 U/ml, 160 U/ml, 170 U/ml, 175 U/ml, 180 U/ml, 190 U/ml, 200 U/ml, 210 U/ml, 220 U/ml, 225 U/ml, 230 U/ml, 240 U/ml, 250 U/ml, 260 U/ml, 270 U/ml, 275 U/ml, 280 U/ml, 290 U/ml, 300 U/ml, 400 U/ml, 500 U/ml, 600 U/ml, 700 U/ml, 800 U/ml, 900 U/ml, 1000 U/ml, or more of the skin care composition.

In an additional embodiment, an active ingredient, including, but not limited to a protein, which includes, but is not limited to a growth factor, contained in a skin care composition should comprise, e.g., about 0.01 pg/ml, about 0.05 pg/ml, about 0.1 pg/ml, about 0.5 pg/ml, about 0.001 ng/ml, about 0.005 ng/ml, about 0.01 ng/ml, about 0.05 ng/ml, about 0.1 ng/ml, about 0.5 ng/ml, about 1 ng/ml, about 1.5 ng/ml, about 2 ng/ml, about 2.5 ng/ml, about 3 ng/ml, about 3.5 ng/ml, about 4 ng/ml, about 4.5 ng/ml, about 5 ng/ml, about 5.5 ng/ml, about 6 ng/ml, about 6.5 ng/ml, about 7 ng/ml, about 7.5 ng/ml, about 8 ng/ml, about 8.5 ng/ml, about 9 ng/ml, about 9.5 ng/ml, about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 30 ng/ml, about 35 ng/ml, about 35 ng/ml, about 40 ng/ml, about 45 ng/ml, about 50 ng/ml, about 55 ng/ml, about 60 ng/ml, about 65 ng/ml, about 65 ng/ml, about 70 ng/ml, about 75 ng/ml, about 80 ng/ml, about 85 ng/ml, about 90 ng/ml, about 95 ng/ml, about 100 ng/mL, about 105 ng/ml, about 110 ng/ml, about 115 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, about 200 ng/ml, about 205 ng/ml, about 210 ng/ml, about 215 ng/ml, about 220 ng/ml, about 225 ng/ml, about 230 ng/ml, about 235 ng/ml, about 240 ng/ml, about 245 ng/ml, about 250 ng/ml, about 250 ng/ml, about 255 ng/ml, about 260 ng/ml, about 265 ng/ml, about 270 ng/ml, about 275 ng/ml, about 280 ng/ml, about 285 ng/ml, about 290 ng/ml, about 295 ng/ml, about 300 ng/mL 305 ng/ml, about 310 ng/ml, about 315 ng/ml, about 320 ng/ml, about 325 ng/ml, about 330 ng/ml, about 335 ng/ml, about 340 ng/ml, about 345 ng/ml, about 350 ng/ml, about 355 ng/ml, about 360 ng/ml, about 365 ng/ml, about 370 ng/ml, about 375 ng/ml, about 380 ng/ml, about 385 ng/ml, about 390 ng/ml, about 395 ng/ml, about 400 ng/ml, about 500 ng/ml, about 600 ng/ml, about 700 ng/ml, about 800 ng/ml, about 900 ng/ml, about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 11 mg/ml, about 12 mg/ml, about 13 mg/ml, about 14 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 90 mg/ml, about 100 mg/ml, about 125 mg/ml, about 150 mg/ml, about 175 mg/ml, about 200 mg/ml, about 300 mg/ml, about 400 mg/ml, about 500 mg/ml, about 600 mg/ml, about 700 mg/ml, about 800 mg/ml, about 900 mg/ml, about 1 g/ml or more of the skin care composition.

In an additional embodiment, an active ingredient, including, but not limited to a protein, which includes, but is not limited to a growth factor, contained in a skin care composition should comprise, e.g., at least 0.01 pg/ml, at least 0.05 pg/ml, at least 0.1 pg/ml, at least 0.5 pg/ml, at least 0.001 ng/ml, at least 0.005 ng/ml, at least 0.01 ng/ml, at least 0.05 ng/ml, at least 0.1 ng/ml, at least 0.5 ng/ml, at least 1 ng/ml, at least 1.5 ng/ml, at least 2 ng/ml, at least 2.5 ng/ml, at least 3 ng/ml, at least 3.5 ng/ml, at least 4 ng/ml, at least 4.5 ng/ml, at least 5 ng/ml, at least 5.5 ng/ml, at least 6 ng/ml, at least 6.5 ng/ml, at least 7 ng/ml, at least 7.5 ng/ml, at least 8 ng/ml, at least 8.5 ng/ml, at least 9 ng/ml, at least 9.5 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 30 ng/ml, at least 35 ng/ml, at least 35 ng/ml, at least 40 ng/ml, at least 45 ng/ml, at least 50 ng/ml, at least 55 ng/ml, at least 60 ng/ml, at least 65 ng/ml, at least 65 ng/ml, at least 70 ng/ml, at least 75 ng/ml, at least 80 ng/ml, at least 85 ng/ml, at least 90 ng/ml, at least 95 ng/ml, at least 100 ng/mL, at least 105 ng/ml, at least 110 ng/ml, at least 115 ng/ml, at least 120 ng/ml, at least 125 ng/ml, at least 130 ng/ml, at least 135 ng/ml, at least 140 ng/ml, at least 145 ng/ml, at least 150 ng/ml, at least 155 ng/ml, at least 160 ng/ml, at least 165 ng/ml, at least 170 ng/ml, at least 175 ng/ml, at least 180 ng/ml, at least 185 ng/ml, at least 190 ng/ml, at least 195 ng/ml, at least 200 ng/ml, at least 205 ng/ml, at least 210 ng/ml, at least 215 ng/ml, at least 220 ng/ml, at least 225 ng/ml, at least 230 ng/ml, at least 235 ng/ml, at least 240 ng/ml, at least 245 ng/ml, at least 250 ng/ml, at least 250 ng/ml, at least 255 ng/ml, at least 260 ng/ml, at least 265 ng/ml, at least 270 ng/ml, at least 275 ng/ml, at least 280 ng/ml, at least 285 ng/ml, at least 290 ng/ml, at least 295 ng/ml, at least 300 ng/mL 305 ng/ml, at least 310 ng/ml, at least 315 ng/ml, at least 320 ng/ml, at least 325 ng/ml, at least 330 ng/ml, at least 335 ng/ml, at least 340 ng/ml, at least 345 ng/ml, at least 350 ng/ml, at least 355 ng/ml, at least 360 ng/ml, at least 365 ng/ml, at least 370 ng/ml, at least 375 ng/ml, at least 380 ng/ml, at least 385 ng/ml, at least 390 ng/ml, at least 395 ng/ml, at least 400 ng/ml, at least 500 ng/ml, at least 600 ng/ml, at least 700 ng/ml, at least 800 ng/ml, at least 900 ng/ml, at least 1 mg/ml, at least 2 mg/ml, at least 3 mg/ml, at least 4 mg/ml, at least 5 mg/ml, at least 6 mg/ml, at least 7 mg/ml, at least 8 mg/ml, at least 9 mg/ml, at least 10 mg/ml, at least 11 mg/ml, at least 12 mg/ml, at least 13 mg/ml, at least 14 mg/ml, at least 15 mg/ml, at least 20 mg/ml, at least 25 mg/ml, at least 30 mg/ml, at least 35 mg/ml, at least 40 mg/ml, at least 45 mg/ml, at least 50 mg/ml, at least 55 mg/ml, at least 60 mg/ml, at least 65 mg/ml, at least 70 mg/ml, at least 75 mg/ml, at least 80 mg/ml, at least 90 mg/ml, at least 100 mg/ml, at least 125 mg/ml, at least 150 mg/ml, at least 175 mg/ml, at least 200 mg/ml, at least 300 mg/ml, at least 400 mg/ml, at least 500 mg/ml, at least 600 mg/ml, at least 700 mg/ml, at least 800 mg/ml, at least 900 mg/ml, at least 1 g/ml or more of the skin care composition.

In a further embodiment, an active ingredient, including, but not limited to a protein, which includes, but is not limited to a growth factor, contained in a skin care composition has a concentration in the range of, e.g., about 0.001 pg/ml to about 75 ng/ml, about 0.01 pg/ml to about 75 ng/ml, about 0.1 pg to about 75 ng/ml, about 1.0 pg to about 75 ng/ml, about 5 ng/ml to about 75 ng/ml, about 10 ng/ml to about 75 ng/ml, about 15 ng/ml to about 75 ng/ml, about 20 ng/ml to about 75 ng/ml, about 25 ng/ml to about 75 ng/ml, about 30 ng/ml to about 75 ng/ml, about 35 ng/ml to about 75 ng/ml, about 40 ng/ml to about 75 ng/ml, about 50 ng/ml to about 75 ng/ml, about 0.001 pg/ml to about 100 ng/ml, about 0.01 pg/ml to about 100 ng/ml, about 0.1 pg to about 100 ng/ml, about 1.0 pg to about 100 ng/ml, about 5 ng/ml to about 100 ng/ml, about 10 ng/ml to about 100 ng/ml, about 15 ng/ml to about 100 ng/ml, about 20 ng/ml to about 100 ng/ml, about 25 ng/ml to about 100 ng/ml, about 30 ng/ml to about 100 ng/ml, about 35 ng/ml to about 100 ng/ml, about 40 ng/ml to about 100 ng/ml, about 50 ng/ml to about 100 ng/ml, about 0.001 ng/ml to about 400 ng/ml, about 0.01 ng/ml to about 400 ng/ml, about 0.1 ng/ml to about 400 ng/ml, about 1.0 ng/ml to about 400 ng/ml, about 5 ng/ml to about 400 ng/ml, about 10 ng/ml to about 400 ng/ml, about 20 ng/ml to about 400 ng/ml, about 30 ng/ml to about 400 ng/ml, about 40 ng/ml to about 400 ng/ml, about 50 ng/ml to about 400 ng/ml, or about 100 ng/ml to about 1000 ng/ml.

In an embodiment, an active ingredient in a skin care composition is administered to an individual at a dose in the range of about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day of each active ingredient contained in the skin care composition and wherein, a skin care composition is administered to an individual at a dose in the range of about 5 mg/kg/day to about 10 mg/kg/day, about 5 mg/kg/day to about 15 mg/kg/day, about 5 mg/kg/day to about 20 mg/kg/day, about 5 mg/kg/day to about 25 mg/kg/day, about 5 mg/kg/day to about 30 mg/kg/day, about 5 mg/kg/day to about 35 mg/kg/day, about 5 mg/kg/day to about 40 mg/kg/day, about 5 mg/kg/day to about 45 mg/kg/day, about 5 mg/kg/day to about 50 mg/kg/day, about 5 mg/kg/day to about 75 mg/kg/day, or about 5 mg/kg/day to about 100 mg/kg/day of each active ingredient contained in the skin care composition and wherein, a skin care composition is administered to an individual at a dose in the range of 1 mg/day to about 3,000 mg/day of each active ingredient contained in the skin care composition. In a further embodiment, an effective amount of each active ingredient contained in a skin care composition disclosed herein may be, e.g., at least 1 mg/day, at least 5 mg/day, at least 10 mg/day, at least 15 mg/day, at least 20 mg/day, at least 25 mg/day, at least 30 mg/day, at least 40 mg/day, at least 50 mg/day, at least 100 mg/day, at least 150 mg/day, at least 200 mg/day, at least 250 mg/day, at least 300 mg/day, at least 350 mg/day, at least 400 mg/day, at least 450 mg/day, at least 500 mg/day, at least 550 mg/day, at least 600 mg/day, at least 650 mg/day, at least 700 mg/day, at least 750 mg/day, at least 800 mg/day, at least 850 mg/day, at least 900 mg/day, at least 950 mg/day, at least 1,000 mg/day, at least 1,50 mg/day, at least 1,100 mg/day, at least 1,150 mg/day, at least 1,200 mg/day, at least 1,250 mg/day, at least 1,300 mg/day, at least 1,350 mg/day, at least 1,400 mg/day, at least 1,450 mg/day, at least 1,500 mg/day, at least 1,600 mg/day, at least 1,700 mg/day, at least 1,800 mg/day, at least 1,900 mg/day, at least 2,000 mg/day, at least 2,100 mg/day, at least 2,200 mg/day, at least 2,300 mg/day, at least 2,400 mg/day, at least 2,500 mg/day, at least 2,600 mg/day, at least 2,700 mg/day, at least 2,800 mg/day, at least 2,900 mg/day, or at least 3,000 mg/day.

In an embodiment, a skin care composition may comprise an active ingredient, including, without limitation, a biologically active ingredient, in a therapeutically effective amount. In an embodiment, a biologically active ingredient is a protein, which includes, but is not limited to a growth factor. In an embodiment, the term “effective amount” is synonymous with “therapeutically effective amount”, “effective dose”, or “therapeutically effective dose” and when used in reference to maintaining or increasing the health of the skin of an individual and/or rejuvenating the skin of an individual refers to the minimum dose of a therapeutic compound disclosed herein necessary to achieve the desired therapeutic effect and includes a dose sufficient to maintain or increase the health of the skin of an individual and/or rejuvenate the skin of an individual. The effectiveness of a skin care composition disclosed herein capable of maintaining or increasing the health of the skin of an individual and/or rejuvenating the skin of an individual can be determined, without limitation, by observing an improvement in an individual based upon one or more clinical symptoms, and/or physiological indicators associated with maintaining or increasing the health of the skin of an individual and/or rejuvenating the skin of an individual.

In an embodiment, a skin care composition comprises one or more active ingredients, with each active ingredient present at a concentration of at least about 0.1% (w/v), or alternatively at least about 0.01%, 0.02%, 0.05%, 0.075%, 0.2%, 0.3%, 0.4%, 0.5%, 0.6%, 0.7%, 0.8%, 0.9%, 1%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%, 6.25%, 6.5%, 6.75%, 7%, 7.25%, 7.5%, 7.75%, 8%, 8.25%, 8.5%, 8.75%, 9%, 9.25%, 9.5%, 9.75%, 10%, 10.25%, 10.5%, 10.75%, 11%, 11.25%, 11.5%, 11.75%, 12%, 12.25%, 12.5%, 12.75%, 13%, 13.25%, 13.5%, 13.75%, 14%, 14.25%, 14.5%, 14.75%, 15%, 15.25%, 15.5%, 15.75%, 16%, 16.25%, 16.5%, 16.75%, 17%, 17.25%, 17.5%, 17.75%, 18%, 18.25%, 18.5%, 18.75%, 19%, 19.25%, 19.5%, 19.75%, 20%, 20.25%, 20.5%, 20.75%, 21%, 21.25%, 21.5%, 21.75%, 22%, 22.25%, 22.5%, 22.75%, 23%, 23.25%, 23.5%, 23.75%, 24%, 24.25%, 24.5%, 24.75%, 25%, 26%, 27%, 28%, 29%, 30%, 31%, 32%, 33%, 34%, 35%, 40%, or more (w/v) of the skin care composition.

In an embodiment, a skin care composition comprises one or more active ingredients, with each active ingredient present at a concentration of about 0.1% (w/v) to about 40%, or alternatively at about 0.01% to about 25%, 0.02% to about 25%, 0.05% to about 25%, 0.075% to about 25%, 0.2% to about 25%, 0.3% to about 25%, 0.4% to about 25%, 0.5% to about 25%, 0.6% to about 25%, 0.7% to about 25%, 0.8% to about 25%, 0.9% to about 25%, 1% to about 25%, 1.5% to about 25%, 1.75% to about 25%, 2% to about 25%, 2.25% to about 25%, 2.5% to about 25%, 2.75% to about 25%, 3% to about 25%, 3.25% to about 25%, 3.5% to about 25%, 3.75% to about 25%, 4% to about 25%, 4.25% to about 25%, 4.5% to about 25%, 4.75% to about 25%, 5% to about 25%, 5.25% to about 25%, 5.5% to about 25%, 5.75% to about 25%, 6% to about 25%, 6.25% to about 25%, 6.5% to about 25%, 6.75% to about 25%, 7% to about 25%, 7.25% to about 25%, 7.5% to about 25%, 7.75% to about 25%, 8% to about 25%, 8.25% to about 25%, 8.5% to about 25%, 8.75% to about 25%, 9% to about 25%, 9.25% to about 25%, 9.5% to about 25%, 9.75% to about 25%, 10% to about 25%, 10.25% to about 25%, 10.5% to about 25%, 10.75% to about 25%, 11% to about 25%, 11.25% to about 25%, 11.5% to about 25%, 11.75% to about 25%, 12% to about 25%, 12.25% to about 25%, 12.5% to about 25%, 12.75% to about 25%, 13% to about 25%, 13.25% to about 25%, 13.5% to about 25%, 13.75% to about 25%, 14% to about 25%, 14.25% to about 25%, 14.5% to about 25%, 14.75% to about 25%, 15% to about 25%, 15.25% to about 25%, 15.5% to about 25%, 15.75% to about 25%, 16% to about 25%, 16.25% to about 25%, 16.5% to about 25%, 16.75% to about 25%, 17% to about 25%, 17.25% to about 25%, 17.5% to about 25%, 17.75% to about 25%, 18% to about 25%, 18.25% to about 25%, 18.5% to about 25%, 18.75% to about 25%, 19% to about 25%, 19.25% to about 25%, 19.5% to about 25%, 19.75% to about 25%, 20% to about 25%, 20.25% to about 25%, 20.5% to about 25%, 20.75% to about 25%, 5% to about 20%, 6% to about 20%, 7% to about 20%, 8% to about 20%, 9% to about 20%, 10% to about 20%, 11% to about 20%, 12% to about 20%, 13%, to about 20%, 14% to about 20%, 15% to about 20%, 5% to about 15%, 6% to about 15%, 7% to about 15%, 8% to about 15%, 9% to about 15%, 10% to about 15%, 11% to about 15%, 12% to about 15%, 13%, to about 15%, 14% to about 15% (w/v).

In an embodiment, an active ingredient of a skin care composition can be mixed or formulated with a carrier to form of a liquid, a gel suspension, (a semi-solid carrier) ointment, cream, lotion, hydrogel, or a solid carrier to form a paste, a cream, a gel, or other means by which the skin care composition can be applied to an individual's skin; be desiccated and, optionally, mixed with solids to form a powder; or be suspended, dissolved; emulsified; or encapsulated into a variety of physical form, including, without limitation, a liquid or an aerosol for administration to or self-application by an individual. In an embodiment, a skin care composition can be formulated or prepared as a liquid or as a micronized or nanoparticle for aerosol, oral or topical administration, in a form that includes, but is not limited to a spray or aerosolized particle.

In an embodiment, a carrier is a material that does not abrogate the biological activity and properties of the skin care composition. Carriers must be of sufficiently high purity and sufficiently low in terms of irritability, immunogenicity, and toxicity to render them suitable for administration to the mammal being treated. The carrier can be inert, or it can possess pharmaceutical benefits, cosmetic benefits or both.

As used herein the phrase “cosmetically acceptable carrier” refers to a substantially non-toxic carrier, conventionally useable for the topical administration of cosmetics, with which the one or more actives of the present invention will remain stable and bioavailable. It will be understood that cosmetically acceptable carriers and pharmaceutically acceptable carriers are similar, if not often identical, in nature. In the present formulation as disclosed for the promotion of healthy skin, the carrier may be between about 0.0001% (w/v) to about 99% (w/v), 0.0001% (w/v) to about 90% (w/v), 0.0001% (w/v) to about 80% (w/v), about 0.001% (w/v) to about 60.0% (w/v), or about 0.01% (w/v) to about 40.0% (w/v).

In an embodiment, creams or gels may be prepared wherein, without limitation, the active components of the invention are in suspension. Generally, cream bases are categorized into hydrocarbon bases (oleaginous), which may use white petroleum as a base; adsorption bases (anhydrous), which might use hydrophilic petroleum or anhydrous lanolin; emulsion bases (water and oil type); emulsion bases (oil and water type); and water soluble bases, which often use polyethylene glycol as an ointment base.

In an embodiment, liquid suspensions may be formulated, without limitation, by suspending a therapeutic compound disclosed herein in admixture with excipients suitable for the manufacture of aqueous suspensions. In an embodiment, such excipients are suspending agents, for example, without limitation, sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example, without limitation, polyoxyethylene stearate, or condensation products of ethylene oxide with long-chain aliphatic alcohols, for example, without limitation, heptadecaethyleneoxycetanol, or condensation products of ethylene oxide with partial esters derived from fatty acids, for example, without limitation, polyoxyethylene sorbitan monooleate.

Particular excipients as approved for U.S. Food and Drug regulatory purposes can be found at the FDA Inactive Ingredient Database. Many useful excipients are well known in the art and can be found described in, for example, Banga, A. K., Therapeutic Peptides and Proteins, Formulation, Processing and Delivery Systems, (2d Ed 2006, CRC Press), Chapter 4, section 4.4, Pharmaceutical excipients in formulations (at pages 104-116). Any one or more of any other excipients or others may be included in any formulation as described herein. Similarly, in an embodiment, at least one excipient can confer more than one of the functions onto a formulation. Alternatively, in another embodiment, two or more excipients can be included in a formulation to perform more than one of the above or other functions. For example, an excipient can, without limitation, be included as a component in a formulation to change, adjust or optimize the osmolality of the formulation, thereby acting as a tonicity modifier.

In an embodiment, a cosmeceutically active composition of the present invention can be formulated as an emulsion for topical application. An emulsion contains a first liquid distributed into the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, and buffers.

In an embodiment, non-limiting representative examples of components (also called exicipients or adjuvants) present as, or in addition to carriers, include; a moisturizing agent or humectant, a pH adjusting agent, a deodorant or odor absorbing agent, a fragrance, a chelating agent, an emulsifing agent, thickeners, solubilizing agents, penetration enhancers, colorants, UV absorbents, an anti-oxidant agent, and a surfactant.

In an embodiment, a suitable pharmaceutically or cosmetically acceptable carrier include water, petroleum jelly, petroleum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, and the like. In an aspect of the invention the polymer is selected from a carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polyethylene glycol (PEG), branched PEG, PolyPEG®, polysialic acid (PSA), starch, hydroxyalkyl starch (HAS), hydroxylethyl starch (HES), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammonium phosphate (MPC).

In an embodiment, the protein can be an extended half-life form. Extended half-life forms can be prepared by linking a water soluble polymer to a protein through either a stable or a releasable linkage. In an embodiment, an extended half-life form is a fusion protein, a truncated protein, a protein with a modified carbohydrate pattern, a protein wherein amino acids have been replaced or other normative protein. In an embodiment, the water soluble polymer is, without limitation selected from the group consisting of carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polyethylene glycol (PEG), branched PEG, PolyPEG®, polysialic acid (PSA), starch, hydroxyalkyl starch (HAS), hydroxylethyl starch (HES), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammonium phosphate (MPC). In an embodiment the protein-water soluble polymer conjugate has a biological activity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, or 150 percent (%) biological activity relative to native unmodified protein. In a further embodiment, the water soluble polymer is from about 1,000 kD to about 150,000 kD, from about 2,000 kD to about 125,000 kD, from about 3,000 kD to about 100,000 kD, from about 4,000 kD to about 100,000 kD, from about 5,000 kD to about 100,000 kD, from about 10,000 kD to about 100,000 kD, from about 15,000 kD to about 100,000 kD, from about 20,000 kD to about 100,000 kD, from about 25,000 kD to about 100,000 kD, from about 30,000 kD to about 100,000 kD, from about 35,000 kD to about 100,000 kD, from about 40,000 kD to about 100,000 kD, from about 50,000 kD to about 1000,000 kD. In an embodiment the water soluble polymer is at least 250 kD, 500 kD, 750 kD, 1000 kD, 1,250 kD, 1500 kD, 1,750 kD, 2,000 kD, 2,500 kD, 3,000 kD, 3,500 kD, 4,000 kD, 4,500 kD, 5,000 kD 5,500 kD, 6,000 kD, 6,500 kD, 7,000 kD, 7,500 kD, 8,000 kD, 8,500 kD, 9,000 kD, 9500 kD, 10,000 kD, 11,000 kD, 12,000 kD, 13,000 kD, 14,000 kD, 15,000 kD, 16,000 kD, 17,000 kD, 18,000 kD, 19,000 kD, 20,000 kD, 25,000 kD, 30,000 kD, 35,000 kD, 40,000 kD, 45,000 kD, 50,000 kD, 60,000 kD, 70,000 kD, 80,000 kD, 90,000 kD, 100,00 kD, 110,000 kD, 120,000 kD, 130,000 kD, 140,000 kD, 150,000 kD or any combination thereof.

In an embodiment a representative examples of a moisturizing or humectant agent that are usable in the present invention include to add or restore moisture to the skin are, without limitation, guanidine, glycolic acid and glycolate salts (e.g. ammonium salt and quaternary alkyl ammonium salt), aloe vera in any of its variety of forms (e.g., aloe vera gel), allantoin, argan oil (such as a preparation or extract of bark or seeds of the argan tree), urazole, polyhydroxy alcohols such as sorbitol, glycerol, hexanetriol, propylene glycol, butylene glycol, hexylene glycol and the like, polyethylene glycols, simple and complex saccharides and polysaccharides and derivatives (e.g., alkoxylated glucose), hyaluronic acid, lactamide monoethanolamine, acetamide monoethanolamine and any combination thereof.

As the skin is naturally acidic having a measurable pH of 5.5, compositions for topical skin application (to avoid irritation), in an embodiment, without limitation, have a pH value of between 4.0 and 7.0. In an embodiment, a skin care composition is formulated to have a pH value that ranges between about 4.0 and about 7.0, between about 5.0 and about 6.0. In any of the above embodiments of the formulations, whether containing one or two or more growth factor types and additional cosmetically or pharmaceutically active agents, may have a pH that is from about 4 to about 6 or from about 5 to about 7.5. Alternatively, in a further embodiment, without limitation, the pH is from about 5 to about 6, from about 6 to about 7, or from about 6.5 to about 7.5. In an embodiment, the pH is at least about 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12. In a further embodiment, the pH of the formulation is in the range of about 2 to about 12, about 3 to about 11, about 4 to about 10, about 5 to about 9, about 6 to about 8, about 6 to about 7, about 6 to about 9, about 6 to about 10, about 5 to about 6, about 5 to about 7, about 5 to about 8, about 4 to about 9, about 4 to about 8, about 4 to about 7, about 4 to about 6, about 4 to about 5, about 3 to about 10, about 3 to about 9, about 3 to about 8, about 3 to about 7, about 3 to about 6, about 3 to about 5, about 3 to about 4, about 2 to about 11, about 2 to about 10, about 2 to about 9, about 2 to about 8, about 2 to about 7, about 2 to about 6, about 2 to about 5, about 2 to about 4, about 2 to about 3, about 7 to about 8, about 7 to about 9, about 7 to about 10, about 7 to about 11, about 7 to about 12, about 8 to about 9, about 8 to about 10, about 8 to about 11, about 8 to about 12, about 9 to about 10, about 9 to about 11, about 9 to about 12, about 10 to about 11, about 10 to about 12 or about 11 to about 12. Hence, a pH adjusting agent is typically added to bring the pH of the composition to the desired value. Suitable pH adjusting agents include, for example, one or more low molecular weight organic acids, such as adipic acid, glycine, citric acid; salts such as calcium or sodium hydroxide, magnesium aluminometasilicates, phosphate buffers or any combinations thereof.

In an embodiment, a representative example of a deodorant or an odor masking agent that is usable in the context of the present invention include, without limitation, quaternary ammonium compounds such as cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmityl sarcosine, lauroyl sarcosine, N-myristoyl glycine, potassium N-lauryl sarcosine, stearyl, trimethyl ammonium chloride, sodium aluminum chlorohydroxy lactate, tricetylmethyl ammonium chloride, 2,4,4′-trichloro-2′-hydroxy diphenyl ether, diaminoalkyl amides such as L-lysine hexadecyl amide, heavy metal salts of citrate, salicylate, and piroctose, especially zinc salts, and acids thereof, heavy metal salts of pyrithione, especially zinc pyrithione and zinc phenolsulfate. In another embodiment, an odor absorbing material includes carbonate and bicarbonate salts, e.g. as the alkali metal carbonates and bicarbonates, ammonium and tetraalkylammonium carbonates and bicarbonates, and includes, without limitation, the sodium and potassium salts, or any combination of the above. In another embodiment, it will be recognized that some salts of organic or inorganic acids may serve dual functions as buffering agents and odor masking or deodorant agents.

In an embodiment, a chelating agent is, without limitation a multifunctionalized compound capable of forming coordination complex with, e.g. a multivalent metal ion. In a further embodiment, a chelating agent is optionally added to the compositions of the present invention so as to enhance the preservative or preservative system and stability of the preparation. In another embodiment, a chelating agent includes, but is not limited to, mild agents, such as, for example; ethylenediamine based chelaters, ethylenediamine tetraacetic acid and (EDTA, DPTA), succininc acid, citric acid and derivatives, polyhistidine, polylysine or any combination thereof.

In an embodiment, an emulsifier promotes the formation and stabilization of an emulsion. Natural emulsifying agents may be derived from either animal or vegetable sources. Those from animal sources include, include, without limitation, gelatin, egg yolk, casein, wool fat, or cholesterol. Those from vegetable sources include acacia, tragacanth, chondrus, or pectin. Synthetic agents include, include, without limitation, anionic, cationic or nonionic agents. Particularly useful are sodium lauryl sulfate, benzalkonium chloride or polyethylene glycol 400 monostearate, or any combinations thereof.

In an embodiment, an agent used to increase viscosity include, without limitation, thickeners. In a further embodiment, a thickener can be used include, without limitation, those from plant sources such as cellulose derivatives include methyl cellulose and carboxymethyl cellulose to increase the viscosity, non-ionic water-soluble polymers such as hydroxyethylcellulose, cationic water-soluble polymers such as Polyquat 37 (commercially available under the Trademark SYNTHALEN®), fatty alcohols, fatty acids, anionic polymers and their alkali salts and mixtures thereof, and a polymer as described herein.

In an embodiment, a solubilizing agent is a substance that enables solutes including proteins to dissolve. In another embodiment, a representative solubilizing agent includes, without limitation, complex-forming solubilizers such as citric acid, EDTA, sodium meta-phosphate, succinic acid, urea, cyclodextrin, polyvinylpyrrolidone, diethylammonium-ortho-benzoate, and micelle-forming solubilizers such as polysorbate, e.g., TWEEN® 80. In a further embodiment, a solubilizer usable for the compositions include, without limitation, polyoxyethylene sorbitan, a fatty acid ester, polyoxyethylene n-alkyl ethers, n-alkyl amine n-oxides, polyoxamers, organic solvents such as acetone, phospholipids and cyclodextrins.

In an embodiment, a penetration enhancer is an agent known to accelerate the delivery of a substance through the intact epidermal layers of the skin. In an embodiment, a suitable penetration enhancer includes, but is not limited to, dimethylsulfoxide (DMSO), dimethyl formamide (DMF), allantoin, urazole, N,N-dimethylacetamide (DMA), decylmethylsulfoxide (C₁₀ MSO), polyethylene glycol monolaurate (PEGML), propylene glycol (PG), propylene glycol monolaurate (PGML), glycerol monolaurate (GML), lecithin, alcohols, and the like. In a further embodiment, a permeation enhancer is, without limitation, a vegetable oil, and further include, without limitation, oils including, without limitation, safflower oil, cottonseed oil, sesame oil, and corn oil.

In an embodiment, an anti-oxidant agent is a substance that inhibits oxidation (the loss of electrons or increase in oxidation state of a molecule or functional group) which is reactions which can be promoted by oxygen radicals or peroxides under physiological conditions. In a further embodiment, an anti-oxidant is a vitamin, including, without limitation, vitamins C (ascorbate), vitamin B3 (niacinamide and its derivatives), and E (α-, β-, and γ-tocopherols, tocopherol sorbate, tocopherol acetate, other esters of tocopherol. Phenols, such as butylated hydroxy benzoic acids and their salts, 6-hydroxy-2,5,7,8-tetramethylchroman-2-carboxylic acid (commercially available under the tradename TROLOX®), gallic acid and its alkyl esters, especially propyl gallate are known anti-oxidants. Uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcuminoids (e.g., curcumin, tetrahydrocurcumin, desmethoxycurcumin and bis-desmethoxycurcumin and their esters), lysine, methionine, proline, superoxide dismutase, silymarin, tea extracts, grape skin/seed extracts, melanin, and rosemary extracts are generally regarded as anti-oxidants.

In an embodiment, a surfactant is surface-active substance, including, without limitation, a detergent. In an embodiment, a surfactant includes, but is not limited to, sarcosinate, glutamate, sodium alkyl sulfate, ammonium alkyl sulfate, sodium alkyl sulfate, ammonium alkyl sulfates, ammonium laureth-n-sulfate, sodium laureth-n-sulfate, an isothionate, glycerylether sulfonate, sulfosuccinate, sodium lauroyl sarcosinate, and monosodium lauroyl glutamate, and combinations thereof.

Additional components of the final formulation used as excipients and adjuvants suitable for use in preparing pharmaceutically acceptable carriers and compositions may generally be found in Remington's Pharmaceutical Sciences, 18th or 19th editions, published by the Mack Publishing Company of Easton, Pa. which is incorporated herein by reference.

In an embodiment, a skin care composition includes a colorant. In an embodiment, a colorant includes, but is not limited to, pigments or dyes or a combination thereof as the cosmetic benefit requires. In a further embodiment, a colorant includes, without limitation, iron oxides and titanium oxides. In another embodiment, a colorant includes, without limitation, FD&C approved colorants, D&C approved colorants, and those approved for use in Europe and Japan. See Marmion, D. M., Handbook of US Colorants for Food, Drugs, Cosmetics, and Medical Devices, 3rd ed, 1991 herein incorporated by reference.

In another embodiment, the form of the pharmaceutically or cosmetically acceptable formulation of the present invention includes a sustained release or delayed release carrier. In an embodiment, a carrier can be, without limitation, any material capable of sustained or delayed release of the one or more active ingredients to provide a more efficient administration resulting in less frequent and/or decreased dosage, ease of handling, and extended or delayed effects on epithelial-related conditions. In an embodiment, a carrier can be, without limitation, a liposome, microsponge, microsphere, or microcapsule of natural and synthetic polymers and the like. In a further embodiment, a polypeptide agent is covalently or non-covalently linked to polymers to form a complex which is specifically or non-specifically degraded thereby releasing the active protein during use of the formulation.

In an embodiment, a skin care composition includes a liposome. In a further embodiment, an active ingredient of a skin care composition is encapsulated in a liposome. In another embodiment, an active ingredient of a skin care composition is associated with the outside of a liposome. In an embodiment an active ingredient is associated with a liposome through either, without limitation, an electrostatic interaction, van Der Waal interaction, covalent coupling and/or non-covalent coupling. In another embodiment, two or more active ingredients are encapsulated in a liposome. In an embodiment, two or more active ingredients are associated with a liposome. For purposes of this invention, liposomes include, without limitation, multivesicular liposomes (MVL), which are man-made, microscopic lipid vesicles comprising lipid membranes enclosing multiple non-concentric aqueous chambers. In a further embodiment, a liposome is, without limitation, a multilamellar liposome or vesicle (MLV), which have multiple “onion-skin” concentric membranes, in between which are shell-like concentric aqueous compartments.

In an embodiment, a liposome has, without limitation, a mean diameter in the micrometer range, usually from 0.5 to 25 micrometer. In an embodiment, a liposome has a diameter of at least 0.1 μm, 0.2 μm, 0.3 μm, −0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 35 μm, 40 μm, 55 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 225 μm, 250 μm, 275 μm, 300 μm, 325 μm, 350 μm, 375 μm, 400 μm, 425 μm, 450 μm, 475 μm, 500 μm, 600 μm, 700 μm, 800 μm, 900 μm, 1000 μm, or more

Multilamellar and unilamellar liposomes can be made by several relatively simple methods. The prior art describes a number of techniques for producing ULV and MLV (for example U.S. Pat. No. 4,522,803 to Lenk; U.S. Pat. No. 4,310,506 to Baldeschweiler; U.S. Pat. No. 4,235,871 to Papahadjopoulos; U.S. Pat. No. 4,224,179 to Schneider, U.S. Pat. No. 4,078,052 to Papahadjopoulos; U.S. Pat. No. 4,394,372 to Taylor U.S. Pat. No. 4,308,166 to Marchetti; U.S. Pat. No. 4,485,054 to Mezei; and U.S. Pat. No. 4,508,703 to Redziniak).

A formulation can, in general, be prepared according to pharmaceutical or cosmetic standards and using approved grades of reagents. A formulation can be prepared using sterile reagents in a sterile manufacturing environment or sterilized following preparation. Sterile solutions for oral administration, topical application or injection can be prepared using known procedures in the art including, for example, by incorporating one or more bioactive agents in the required amount in with a carrier or excipient as described herein followed by sterilization microfiltration or vacuum drying and freeze-drying (lyophilization) a sterile composition under sterile conditions.

In an embodiment, a cosmetically acceptable carrier is, without limitation, described in the CTFA International Cosmetic Ingredient Dictionary and Handbook, 8th edition, edited by Wenninger and Canterbery, (The Cosmetic, Toiletry, and Fragrance Association, Inc., Washington, D.C., 2000), which is herein incorporated by reference. Also included are the carriers described hereinabove.

In an embodiment, a “stable composition” or “stable formulation” or “shelf stable formulation or “stable pharmaceutical formulation” or “stable cosmetic formulation” as used in connection with the compositions and formulations described herein denotes, without limitation, a composition or formulation, which preserves its physical stability/identity/integrity and/or chemical stability/identity/integrity and/or biological activity/identity/integrity during manufacturing, storage and application. Various analytical techniques for evaluating protein stability are available in the art and reviewed in Reubsaet, J. L., J. H. Beijnen, et al. (1998) “Analytical techniques used to study the degradation of proteins and peptides: chemical instability,” J Pharm Biomed Anal 17(6 7): 955 78 and Wang, W. (1999) “Instability, stabilization, and formulation of liquid protein pharmaceuticals,” Int J Pharm 185(2): 129 88. In an embodiment, stability is evaluated by, for example, without limitation, storage at selected climate conditions for a selected time period, by applying mechanical stress such as shaking at a selected shaking frequency for a selected time period, by irradiation with a selected light intensity for a selected period of time, or by repetitive freezing and thawing at selected temperatures. In an embodiment, stability is determined by, for example, without limitation, at least one of the methods selected from the group consisting of visual inspection, SDS-PAGE, IEF, size exclusion liquid chromatography (SEC-HPLC), reversed phase liquid chromatography (RP-HPLC), ion-exchange HPLC, capillary electrophoresis, light scattering, particle counting, turbidity, RFFIT, and kappa/lambda ELISA, without limitation.

In an embodiment, a composition or formulation disclosed herein is considered stable when the protein in the composition or formulation (1) retains its physical stability, (2) retains its chemical stability and/or (3) retains it biological activity. In an embodiment, a protein may be said to “retain its physical stability” in a composition or formulation if, for example, without limitation, it shows no signs of aggregation, precipitation and/or denaturation upon visual examination of color and/or clarity, or as measured by UV light scattering or by size exclusion chromatography (SEC) or electrophoresis, such as with reference to turbidity or aggregate formation. In aspects of this embodiment, a protein disclosed herein retains its physical stability in a composition or formulation disclosed herein for a time period of, e.g., at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, at least 24 months, at least 30 months, at least 36 months, at least 40 months, at least 44 months, or at least 48 months.

In other aspects of this embodiment, a protein disclosed herein retains its physical stability in a composition or formulation disclosed herein for a time period of, e.g., about 3 months to about 6 months, about 3 months to about 9 months, about 3 months to about 12 months, about 3 months to about 15 months, about 3 months to about 18 months, about 3 months to about 21 months, about 3 months to about 24 months, about 3 months to about 27 months, about 3 months to about 30 months, about 3 months to about 33 months, about 3 months to about 36 months, about 3 months to about 39 months, about 3 months to about 42 months, about 3 months to about 45 months, about 3 months to about 48 months, about 6 months to about 9 months, about 6 months to about 12 months, about 6 months to about 15 months, about 6 months to about 18 months, about 6 months to about 21 months, about 6 months to about 24 months, about 6 months to about 27 months, about 6 months to about 30 months, about 6 months to about 33 months, about 6 months to about 36 months, about 6 months to about 39 months, about 6 months to about 42 months, about 6 months to about 45 months, about 6 months to about 48 months, about 9 months to about 12 months, about 9 months to about 15 months, about 9 months to about 18 months, about 9 months to about 21 months, about 9 months to about 24 months, about 9 months to about 27 months, about 9 months to about 30 months, about 9 months to about 33 months, about 9 months to about 36 months, about 9 months to about 39 months, about 9 months to about 42 months, about 9 months to about 45 months, about 9 months to about 48 months, about 12 months to about 15 months, about 12 months to about 18 months, about 12 months to about 21 months, about 12 months to about 24 months, about 12 months to about 27 months, about 12 months to about 30 months, about 12 months to about 33 months, about 12 months to about 36 months, about 12 months to about 39 months, about 12 months to about 42 months, about 12 months to about 45 months, about 12 months to about 48 months, about 15 months to about 18 months, about 15 months to about 21 months, about 15 months to about 24 months, about 15 months to about 27 months, about 15 months to about 30 months, about 15 months to about 33 months, about 15 months to about 36 months, about 15 months to about 39 months, about 15 months to about 42 months, about 15 months to about 45 months, about 15 months to about 48 months, about 18 months to about 21 months, about 18 months to about 24 months, about 18 months to about 27 months, about 18 months to about 30 months, about 18 months to about 33 months, about 18 months to about 36 months, about 18 months to about 39 months, about 18 months to about 42 months, about 18 months to about 45 months, about 18 months to about 48 months, about 21 months to about 24 months, about 21 months to about 27 months, about 21 months to about 30 months, about 21 months to about 33 months, about 21 months to about 36 months, about 21 months to about 39 months, about 21 months to about 42 months, about 21 months to about 45 months, about 21 months to about 48 months, about 24 months to about 27 months, about 24 months to about 30 months, about 24 months to about 33 months, about 24 months to about 36 months, about 24 months to about 39 months, about 24 months to about 42 months, about 24 months to about 45 months, about 24 months to about 48 months, about 30 months to about 36 months, about 30 months to about 42 months, about 30 months to about 48 months, about 36 months to about 42 months, about 36 months to about 48 months, or about 42 months to about 48 months.

In an embodiment, a protein may be said to “retain its chemical stability” in a composition or a formulation disclosed herein, if, for example, without limitation, the chemical stability at a given time is such that there is no significant modification of the protein by bond formation or cleavage resulting in a new chemical entity. In a further embodiment, chemical stability can be assessed by detecting and quantifying chemically altered forms of the protein. Chemical alteration may involve, example, without limitation, size modification (e.g. clipping) which can be evaluated using size exclusion chromatography, SDS-PAGE and/or matrix-assisted laser desorption ionization/time-of-flight mass spectrometry (MALDI/TOF MS). Other types of chemical alteration include, for example, without limitation, charge alteration (e.g. occurring as a result of deamidation), which can be evaluated by ion-exchange chromatography, for example. Oxidation is another commonly seen chemical modification. In aspects of this embodiment, a protein disclosed herein retains its chemical stability in a composition or formulation disclosed herein for a time period of, e.g., at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, at least 24 months, at least 30 months, at least 36 months, at least 40 months, at least 44 months, or at least 48 months.

In other aspects of this embodiment, a protein disclosed herein retains its chemical stability in a composition or formulation disclosed herein for a time period of, e.g., about 3 months to about 6 months, about 3 months to about 9 months, about 3 months to about 12 months, about 3 months to about 15 months, about 3 months to about 18 months, about 3 months to about 21 months, about 3 months to about 24 months, about 3 months to about 27 months, about 3 months to about 30 months, about 3 months to about 33 months, about 3 months to about 36 months, about 3 months to about 39 months, about 3 months to about 42 months, about 3 months to about 45 months, about 3 months to about 48 months, about 6 months to about 9 months, about 6 months to about 12 months, about 6 months to about 15 months, about 6 months to about 18 months, about 6 months to about 21 months, about 6 months to about 24 months, about 6 months to about 27 months, about 6 months to about 30 months, about 6 months to about 33 months, about 6 months to about 36 months, about 6 months to about 39 months, about 6 months to about 42 months, about 6 months to about 45 months, about 6 months to about 48 months, about 9 months to about 12 months, about 9 months to about 15 months, about 9 months to about 18 months, about 9 months to about 21 months, about 9 months to about 24 months, about 9 months to about 27 months, about 9 months to about 30 months, about 9 months to about 33 months, about 9 months to about 36 months, about 9 months to about 39 months, about 9 months to about 42 months, about 9 months to about 45 months, about 9 months to about 48 months, about 12 months to about 15 months, about 12 months to about 18 months, about 12 months to about 21 months, about 12 months to about 24 months, about 12 months to about 27 months, about 12 months to about 30 months, about 12 months to about 33 months, about 12 months to about 36 months, about 12 months to about 39 months, about 12 months to about 42 months, about 12 months to about 45 months, about 12 months to about 48 months, about 15 months to about 18 months, about 15 months to about 21 months, about 15 months to about 24 months, about 15 months to about 27 months, about 15 months to about 30 months, about 15 months to about 33 months, about 15 months to about 36 months, about 15 months to about 39 months, about 15 months to about 42 months, about 15 months to about 45 months, about 15 months to about 48 months, about 18 months to about 21 months, about 18 months to about 24 months, about 18 months to about 27 months, about 18 months to about 30 months, about 18 months to about 33 months, about 18 months to about 36 months, about 18 months to about 39 months, about 18 months to about 42 months, about 18 months to about 45 months, about 18 months to about 48 months, about 21 months to about 24 months, about 21 months to about 27 months, about 21 months to about 30 months, about 21 months to about 33 months, about 21 months to about 36 months, about 21 months to about 39 months, about 21 months to about 42 months, about 21 months to about 45 months, about 21 months to about 48 months, about 24 months to about 27 months, about 24 months to about 30 months, about 24 months to about 33 months, about 24 months to about 36 months, about 24 months to about 39 months, about 24 months to about 42 months, about 24 months to about 45 months, about 24 months to about 48 months, about 30 months to about 36 months, about 30 months to about 42 months, about 30 months to about 48 months, about 36 months to about 42 months, about 36 months to about 48 months, or about 42 months to about 48 months.

In an embodiment, a protein may be said to “retain its biological activity” relative to native unmodified protein in a composition or a formulation disclosed herein. In aspects of this embodiment, a protein disclosed herein retain its biological in a composition or formulation disclosed herein when the biological activity present is, e.g., at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In other aspects of this embodiment, a protein disclosed herein retain its biological in a composition or formulation disclosed herein when the biological activity present is between, e.g., about 50% to about 200%, about 55% to about 190%, about 60% to about 170%, about 65% to about 160%, about 70% to about 150%, about 75% to about 140%, about 80% to about 130%, about 85% to about 120%, about 90% to about 110%, about 95% to about 105%, about 50% to about 100%, about 55% to about 100%, about 60% to about 100%, about 65% to about 100%, about 70% to about 100%, about 75% to about 100%, about 80% to about 100%, about 85% to about 100%, about 90% to about 100%, or about 95% to about 100%.

In an embodiment, a skin care composition contains one or more active ingredients that are, without limitation, an extended release ingredients, a sustained release ingredients, a long acting ingredients, an immediate release ingredients, a slow release or controlled release ingredients and wherein, an active ingredient of the skin care composition is released over a period of about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In an embodiment, one or more active ingredients of a skin care composition are released over a period of at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration and wherein, one or more active ingredients of a skin care composition are released over a period of about 1 day after administration, about 2 days after administration, about 3 days after administration, about 4 days after administration, about 5 days after administration, about 6 days after administration or about 7 days or more after administration.

In an embodiment, a skin care composition is capable of maintaining or increasing the health of the skin of an individual and/or rejuvenate the skin of an individual by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% and a skin care composition is capable of maintaining or increasing the health of the skin of an individual and/or rejuvenate the skin of an individual by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

In an embodiment, a skin care composition includes, without limitation, other acceptable compounds, including, without limitation, buffers, preservatives, tonicity adjusters, salts, antioxidants, osmolality adjusting agents, physiological substances, pharmacological substances, bulking agents, emulsifying agents, wetting agents, scenting agents, coloring agents, and the like. In an embodiment, various buffers and means for adjusting pH can be used to prepare a skin care composition provided that the resulting preparation is pharmacologically acceptable. In an embodiment, such buffers include, without limitation, acetate buffers, citrate buffers, phosphate buffers, neutral buffered saline, phosphate buffered saline and borate buffers. It is understood that acids or bases can be used to adjust the pH of a composition as needed. In a further embodiment, pharmacologically acceptable antioxidants include, without limitation, sodium metabisulfite, sodium thiosulfate, acetylcysteine, butylated hydroxyanisole and butylated hydroxytoluene. In an embodiment, useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. In a further embodiment, tonicity adjustors useful in a skin care composition include, without limitation, salts such as, e.g., sodium chloride, potassium chloride, mannitol or glycerin and other pharmaceutically acceptable tonicity adjustor.

In an embodiment, an “international unit” or “IU” of EPO activity is defined as the amount of EPO giving the same amount of erythroid stimulus as 5 microgram of cobalt. Cobalt, a naturally-occurring element with properties similar to those of iron and nickel, induces a marked and stable polycythemic response through transcription of the erythropoietin gene. The international reference standard for EPO assays use isolated human urinary EPO. EPO standards are calibrated against reference EPO preparations, in particular, the Second International Standard for Recombinant-Derived EPO supplied by the World Health Organization (WHO) or the National Institute for Biological Standards and Control (NIBSC). Units of activity are defined as the amount of EPO that gives the same amount of erythroid stimulation as 5 micromoles of cobalt. However, usually EPO preparations are calibrated in bioassays against a reference standard. Human urinary EPO typically has a specific activity of about 70,000 U/mg of protein while values reported for human recombinant EPO may range between 100,000 to 200,000 IU/mg depending on the carbohydrate (glycosylation) content of the product. EPO amounts are expressed in units (U) rather than in grams or moles, because native EPO and rhEPOs are mixtures of isoforms with differing bioactivities. Accordingly, per definition, one EPO unit elicits the same erythropoiesis-stimulating response in rodents (historically: fasted rats) as five micromoles of cobaltous chloride.

Other in vivo and in vitro assays can be used to assess the amount of biological activity. For example, erythropoietic activity can be measured in vitro in the short term culture of cell lines of hematopoietic lineage, e.g. bone marrow or spleen derived cells (FDC-P1/ER, a well characterized nontransformed murine bone marrow derived cell line in which EPO-R has been stably transfected (Dexter, et al., 1980 J. Exp. Med. 152:1036-1047), or EPO responsive tumor cell lines such as TF1 (Kitamura, et al., 1989 Blood 73:375-380) or UT7 cells (Kitamura et al. 1989. J Cell Physiol. 140:323; Komatsu, N., et al. Blood 82(2), 456-464, 1993), or cell lines engineered to be dependent upon EPO for growth. An assay includes, without limitation, the UT7 cell proliferation assay for selection of the concentration of an ESA to formulate to achieve an effect on skin improvement. The EC50 is calculated from a curve fit of concentration vs proliferation as measured by the increase in absorbance of the chromophore or other signal. The EC50 for unmodified EPO is approximately 1.8.times.10⁻¹¹ M. Erythropoietin (EPO) is a heavily glycosylated protein consisting of 165 amino acids with a molecular weight of about 30,000-34,000 Daltons. Knowing these values, UT7 units for other agents can be standardized to EPO, e.g. rhEPO has about 0.5 to about 0.6 UT7 units per ng.

EGF, IGF, and TGFβ responsive cells lines are also known and can be employed in a similar manner to identify standardized or operational units of activity per unit of weight of a preparation comprising the protein. The unit activity can be used in addition to or instead of the weight per unit volume (e.g. ng/ml) or weight per unit weight (mg/gm) or percent weight (e.g. 0.0001% by weight or ppm where 1 ppm is equivalent to 1/10̂6). For example, for a commercial preparation of IGF-1 judged 98% pure by PAGE and HPLC analysis, the ED₅₀ was determined by a cell proliferation assay using FDC-P1 cells is ≦2.0 ng/ml, corresponding to a specific activity of ≧5×10⁵ units/mg. For a commercial human EGF preparation judged 98% pure by PAGE and HPLC analysis, the ED₅₀ was determined by a cell proliferation assay using balb/c 3T3 cells is ≦0.1 ng/ml, corresponding to a specific activity of ≧1×10⁷ units/mg. For human TGFβ3 a commercial preparation judged 98% pure by PAGE and HPLC analysis, the ED₅₀ was determined by TGFβ3's ability to inhibit the mouse IL-4-dependent proliferation of mouse HT-2 cells. The expected ED₅₀ is ≦0.05 ng/ml, corresponding to a specific activity of ≧2×10⁷ units/mg.

Where the composition of the invention is based on units of activity, such as cell proliferation based assay units (U), the composition of the invention comprises between 0.0001 U/ml and about 100 U/ml, between 0.001 U/ml and about 10 U/ml, between 0.01 U/ml and about 100 U/ml, between 0.1 U/ml and about 100 U/ml, between 1 U/ml and about 100 U/ml, between 0.01 U/ml and about 1000 U/ml, between 0.1 U/ml and about 500 U/ml, between 1 U/ml and about 100 U/ml.

In an embodiment, a skin care composition is used to maintain or improve the health of an individual, including, without limitation, preventing, ameliorating, or repairing the effects of aging or effects due to internal or environmental stress on the skin. In a further embodiment, the skin care composition is used, without limitation, to promote the improvement or the appearance, a feature or a function of the skin in an individual in need thereof. In another embodiment, the skin care composition is used, without limitation, to rejuvenate the skin of an individual, including, without limitation, the stimulation of cell growth and cell replacement in the skin of an individual and/or the stimulation of the growth of blood vessels and other means of replenishing the skin of an individual.

In embodiment, a skin feature or function to be improved is fine lines and wrinkles; age spots and dyspigmentation; decreased skin texture, tone and elasticity; roughness, photodamage; abnormal skin epidermal thickness; decreased skin thickness; decreased smoothness, tightness of skin; age spots; fine and coarse lines and wrinkles; fine and coarse periorbital wrinkles; deeper or more abundant nasolabial folds; facial fine and coarse lines; decreased skin radiance, decreased evenness of color or pigmentation; decreased skin firmness; hyperpigmentation; dark spots and/or patches; decreased skin brightness and healthy appearance; intrinsically and extrinsically aged skin; abnormal skin cellular turnover; decreased skin barrier; decreased skin hydration or ability to retain water; brown and red blotchiness; redness; reduction of dermal epidermal junction; loss of density or individual thickness of hair; increased pore size and number of pores; or a combination thereof.

In an embodiment, a skin care composition improves a skin feature or function by a subjective or objectively amount by about 1% to about 100%, about 2% to about 98%, about 5% to about 90%, about 5% to about 80%, about 5% to about 70%, about 5% to about 60%, about 5% to about 40%, about 5% to about 30%, about 5% to about 20%, about 10% to about 80%, about 10% to about 70%, about 10% to about 60%, about 10% to about 50%, about 10% to about 40%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50% and/or at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% or at least 100% or at least 110% or at least 125% or at least 150% or at least 175% or at least 200% or at least 300% or at least 400% or at least 500%.

In an embodiment, administration of a skin care composition described herein may result in more than a 100% such as about 125%, about 150%, about 200% or more improvement and, wherein, without limitation, improvement can also be expressed as a 2-fold improvement of one or more features or functions. In a further embodiment, a skin care composition results in a folds improvement of one or more features or functions include, but are not limited to, 3-fold, 5-fold, 10-fold, 15-fold, 20-fold, 25-fold, 75-fold, 100-fold or more, or any number there between.

In an embodiment, for treatment of a individual, the effective amount of the skin care composition may vary with the particular area of skin being treated, the age and physical condition of the biological individual being treated, the severity of the condition, the duration and frequency of the treatment, the nature of concurrent therapy, the specific compound, composition or other active ingredient employed, the particular carrier utilized, and like factors.

In an embodiment, a skin care composition is used, without limitation, for caring for the skin, by applying an effective amount of the skin care composition to the skin of an individual. In a further embodiment, the amount of a skin care composition applied and the schedule of applying a skin care composition will depend, without limitation, on the exact effect desired to be achieved. In a further embodiment, a skin care composition is applied to the skin so that the one or more of the biologically active proteins selected from an EGFP, an ESA, an IGF, a TGFβ of the formulation is applied, without limitation, in an amount effective to overcome one or more skin features or functions.

The skin care composition can be applied, without limitation, to the skin, the lips, and/or the scalp in a regimen which includes, without limitation, application of the skin care composition weekly, every other day, daily, twice daily, three or more times daily or more or less frequently to achieve the desired effect. In a further embodiment, application of a skin care composition to the skin, the lips, and/or the scalp may be continued until the desired degree of improvement is achieved or continued indefinitely for preventative purposes. In another embodiment, it may be desirable to apply an active ingredient in one skin care composition, a second active ingredient in a second skin care composition, a third active ingredient in a third skin care composition, a fourth active ingredient in a fourth skin care composition, a fifth active ingredient in a fifth skin care composition, a sixth or more active ingredients in a sixth or more skin care compositions, that is, not in admixture one with another. In another embodiment, two or more active ingredients may be included in a single skin care composition. In a further embodiment, two or more active ingredients may be included in a single skin care composition and two or more active ingredients may be included in a second skin care composition administered to an individual. In an embodiment, a skin care composition may be used concurrently or at time periods separated by from 10 to 1440 minutes or longer, such applying one skin care composition sequentially or one skin care composition may be used more or less frequently than the other so long as the desired effect is achieved. In an embodiment, without limitation, low concentrations, i.e. ranging from 0.1 pg/ml to 1 ng/ml or less, may be used if the cosmetic compositions of the invention are repeatedly administered, i.e. in terms of daily application to skin.

In an embodiment, a skin care composition is applied to an individual, wherein a biologically active protein (a biologically active ingredient) selected from an EGFP, an ESA, an IGF, a TGFβ will be administered at an amount in the range of about 0.0001 ng/ml to about 1 ng/ml, about 0.001 ng/ml to about 1 ng/ml, about 0.01 ng/ml to about 10 ng/ml, about 0.01 ng/day to about 10 ng/day, about 0.01 ng/day to about 15 ng/day, about 0.01 ng/day to about 20 ng/day, about 0.01 ng/day to about 25 ng/day, about 0.01 ng/day to about 30 ng/day, about 0.01 ng/day to about 35 ng/day, about 0.01 ng/day to about 40 ng/day, about 0.01 ng/day to about 45 ng/day, about 0.01 ng/day to about 50 ng/day, about 0.01 ng/day to about 75 ng/day, or about 0.01 ng/day to about 100 ng/day. In another aspect, the composition applied to a individual provides one or more proteins in an amount at least 0.0001 ng/day, at least 0.001 ng/day, at least 0.01 ng/day, at least 0.1 ng/day, at least 1.0 ng/day, at least 5.0 ng/day, at least 10 ng/day, at least 20 ng/day, at least 50 ng/day, at least 100 ng/day, or at least 200 ng/day.

The composition of the present can be formulated as a skin-care product which includes filled contains or compressed forms such as a creams, lotions, powders, gels, foams, oils, sprays, aerosol, mousses, salves, balms, sticks and pencils.

Aspects of the present specification may also be described as follows:
1. A skin care composition comprising two or more active ingredients capable of maintaining or increasing the health of the skin of an individual and/or rejuvenating the skin of an individual.
2. The composition according to embodiment 1, wherein the two or more active ingredients are selected from the group of growth factors, anti-oxidants, curcumins, oils and/or red rice extracts.
3. The composition according to embodiment 2, wherein the growth factor is selected from EGF, Heparin-binding EGF-like growth factor (HB-EGF), transforming growth factor-α (TGFα), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4), TGFβ1, TGFβ2, TGFβ3, inhibin-α, activin-β (forms A-C, E), anti-müllerian hormone, bone morphogenetic proteins (BMP1-11, & 15), and growth and differention factors (GDFs 1-3, 5-11) decapentaplegic, Lefty1, ESA, EPO, IGF1, IGF2, insulin and Nodal.
4. The composition according to embodiment 2, wherein the anti-oxidant is selected from Vitamin C (ascorbate), vitamin B3 (niacinamide and its derivatives), Vitamin E (α-, β-, and γ-tocopherols, tocopherol sorbate, tocopherol acetate, other esters of tocopherol; phenols, such as butylated hydroxy benzoic acids and their salts, 6-hydroxy-2,5,7,8-tetramethylchroman-2-carboxylic acid; gallic acid and its alkyl esters, especially propyl gallate; uric acid and its salts and alkyl esters; sorbic acid and its salts; lipoic acid; amines (e.g., N,N-diethylhydroxylamine, amino-guanidine); sulfhydryl compounds (e.g., glutathione), dihydroxy fumaric acid and its salts; glycine pidolate; arginine pilolate; nordihydroguaiaretic acid; bioflavonoids; curcuminoids (e.g., curcumin, tetrahydrocurcumin, desmethoxycurcumin and bis-desmethoxycurcumin and their esters); lysine; methionine; proline; superoxide dismutase; silymarin; tea extracts; grape skin/seed extracts; melanin; and rosemary extracts.
5. The composition according to embodiment 3, wherein the growth factor is EGF, epoetin (EPO), IGF1, or TGFβ3.
6. The composition according to any one of embodiments 1-3, wherein the first biologically active ingredient is an ESA and the second biologically active ingredient is selected from an EGFP, an IGF, and a TGFβ.
7. The composition according to embodiment 6, wherein the first biologically active ingredient is an ESA and the second biologically active ingredient is an EGFP.
8. The composition according to embodiment 6, wherein the first is an ESA and the second biologically active ingredient is an IGF.
9. The composition according to embodiment 6, wherein the first biologically active ingredient is an ESA and the second biologically active ingredient is a TGFβ.
10. The composition according to embodiment 6, wherein the first biologically active ingredient is an EGFP and the second biologically active ingredient is an IGF.
11. The composition according to embodiment 6, wherein the first biologically active ingredient is an EGFP and the second biologically active ingredient is a TGFβ.
12. The composition according to embodiment 6, wherein the first biologically active ingredient is an IGF and the second biologically active ingredient is a TGFβ.
13. The composition according to any one of embodiments 1-12, further comprising a third biologically active ingredient.
14. The composition according to embodiment 13, wherein the first biologically active ingredient is an ESA, the second biologically active ingredient is EGFP, and the third biologically active ingredient is an IGF.
15. The composition according to embodiment 13, wherein the first biologically active ingredient is an ESA, the second biologically active ingredient is EGFP, and the third biologically active ingredient is a TGFβ.
16. The composition according to embodiment 13, wherein the first biologically active ingredient is an ESA, the second biologically active ingredient is biologically active ingredient an IGF, and the third biologically active ingredient is a TGFβ.
17. The composition according to embodiment 13, wherein the first biologically active ingredient is an EGFP, the second biologically active ingredient is an IGF, and the third biologically active ingredient is a TGFβ.
18. The composition according to any one of embodiments 1-17, further comprising a fourth biologically active ingredient.
19. The composition according to embodiment 18, wherein the first biological active ingredient is that of an EGFP, the second biologically active ingredient is an IGF, and the third biologically active ingredient is a TGFβ, and fourth biologically active ingredient is an ESA or wherein the first biological active ingredient is that of an EGF, the second biological active ingredient is that of an IGF-1, the third biological active ingredient is that of an TGFβ-3, and the fourth biological active ingredient is that of an Erythropoietin-α.
20. The composition according to any one of embodiments 1-17, further comprising an additional active ingredient selected from the group consisting of an anti-inflammatory, an anti-oxidant, and a humectant.
21. The composition according to embodiment 20, comprising a curcuminoid.
22. The composition according to embodiment 20, comprising an ascorbate.
23. The composition according to embodiment 20, comprising a curcuminoid and an ascorbate.
24. The composition according to embodiment 20, comprising a curcumin and an ascorbic acid.
25. The composition according to any one of embodiments 1-24, wherein the biological activity of the active ingredients is expressed in activity units, and wherein each of the activities is present from between about 0.0001 U/ml and about 100 U/ml, between 0.001 U/ml and about 10 U/ml, between 0.01 U/ml and about 100 U/ml, between 0.1 U/ml and about 100 U/ml, between 1 U/ml and about 100 U/ml, between 0.01 U/ml and about 1000 U/ml, between 0.1 U/ml and about 500 U/ml, between 1 U/ml and about 100 U/ml.
26. The composition according to any one of embodiments 2-25, wherein the growth factor is present at in the range of from about 0.01 pg/ml to about 100 ng/ml, from about 0.1 pg to about 100 ng/ml, from about 1.0 pg/ml to about 400 ng/ml, from about 0.001 ng/ml to about 400 ng/ml, from about 0.01 ng/ml to about 400 ng/ml, from about 0.1 ng/ml to about 400 ng/ml, about 1.0 ng/ml to about 400 ng/ml, about 5 ng/ml to about 400 ng/ml, about 10 ng/ml to about 400 ng/ml, about 20 ng/ml to about 400 ng/ml, about 50 ng/ml to about 400 ng/ml, or about 100 ng/ml to about 1000 ng/ml.
27. The composition according to any one of embodiments 2-25, wherein the biological activity is expressed as activity units (U) and the growth factor is present at in the range of from about 0.01 U/ml to about 10 U/ml, about 0.01 U/ml to about 15 U/ml, about 0.01 U/ml to about 20 U/ml, about 0.01 U/ml to about 25 U/ml, about 0.01 U/ml to about 30 U/ml, about 0.01 U/ml to about 35 U/ml, about 0.01 U/ml to about 40 U/ml, about 0.01 U/ml to about 45 U/ml, about 0.01 U/ml to about 50 U/ml, about 0.01 U/ml to about 75 U/ml, or about 0.01 U/ml to about 100 U/ml, at least 0.01 U/ml, at least 0.01 U/ml/day, at least 0.1 U/ml, at least 1.0 U/ml, at least 5.0 U/ml, at least 10 U/ml, at least 20 U/ml, at least 50 U/ml, at least 100 U/ml, or at least 200 U/ml.
28. A formulation comprising the composition as defined in any one of embodiments 1-27, wherein the formulation comprises (a) two or more biologically active ingredient; and (b) a pharmaceutically or cosmetically acceptable carrier.
29. The formulation according to embodiment 28, wherein the carrier is the carrier between about 0.0001% (w/v) to about 99% (w/v), 0.0001% (w/v) to about 90% (w/v), 0.0001% (w/v) to about 80% (w/v), about 0.001% (w/v) to about 60.0% (w/v), or about 0.01% (w/v) to about 40.0% (w/v).
30. The formulation according to embodiment 28 or 29, wherein the carrier is in the form of a liquid, a gel suspension, ointment, cream, lotion, hydrogel, a paste; or a powder.
31. The formulation according to any one of embodiments 28-30, wherein the carrier comprises water, petroleum jelly, petroleum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, and the like. In an aspect of the invention the polymer is selected from a carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, polyacryloylmorpholine, polyvinyl alcohol (PVA), polyethylene glycol (PEG), branched PEG, PolyPEG®, polysialic acid (PSA), starch, hydroxyalkyl starch (HAS), hydroxylethyl starch (HES), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC).
32. The formulation according to any one of embodiments 28-31, wherein the carrier forms an emulsion selected from an oil-in-water emulsion and a water-in-oil emulsion.
33. The formulation according to any one of embodiments 28-32, wherein the carrier forms a liposome.
34. The formulation according to any one of embodiments 28-33, wherein the formulation further comprises one or more of a moisturizing agent or humectant, a pH adjusting agent, a deodorant or odor absorbing agent, a fragrance, a chelating agent, an emulsifing agent, a thickener, a solubilizing agent, a penetration enhancer, a colorant, a UV absorbent, an anti-oxidant agent, and a surfactant.
35. The formulation according to embodiment 34, wherein the moister moisturizing or humectant agents is one or more of guanidine, glycolic acid and glycolate salts (e.g. ammonium salt and quaternary alkyl ammonium salt), aloe vera in any of its variety of forms (e.g., aloe vera gel), allantoin, argan oil (such as a preparation or extract of bark or seeds of the argan tree), urazole, polyhydroxy alcohols such as sorbitol, glycerol, hexanetriol, propylene glycol, butylene glycol, hexylene glycol and the like, polyethylene glycols, simple and complex saccharides and polysaccharides and derivatives (e.g., alkoxylated glucose), hyaluronic acid, lactamide monoethanolamine, acetamide monoethanolamine and any combination thereof.
36. The formulation according to embodiment 34, wherein the pH of the pharmaceutical formulation has a pH value that ranges between about 3.0 and about 12.0, between about 5.0 and about 8.0, from about 4 to about 6, or from about 5 to about 7.5.
37. The formulation according to embodiment 34, wherein the solubilizing agent is one or more of citric acid, EDTA, sodium meta-phosphate, succinic acid, urea, cyclodextrin, polyvinylpyrrolidone, diethylammonium-ortho-benzoate, and micelle-forming solubilizers such as polysorbate, polyoxyethylene sorbitan, a fatty acid ester, polyoxyethylene n-alkyl ethers, n-alkyl amine n-oxides, polyoxamers, organic solvents such as acetone, phospholipids and cyclodextrins.
38. The formulation according to embodiment 34, wherein the surfactant is one or more of a sarcosinate, glutamate, sodium alkyl sulfate, ammonium alkyl sulfate, sodium alkyl sulfate, ammonium alkyl sulfates, ammonium laureth-n-sulfate, sodium laureth-n-sulfate, an isothionate, glycerylether sulfonate, sulfosuccinate, sodium lauroyl sarcosinate, and monosodium lauroyl glutamate.
39. The formulation according to any one of embodiments 28-38, comprising the biologically active protein of embodiment 3, wherein the protein is to be administered to a human or animal at amount of at least 0.01 ng/day, at least 0.01 ng/day, at least 0.1 ng/day, at least 1.0 ng/day, at least 5.0 ng/day, at least 10 ng/day, at least 20 ng/day, at least 50 ng/day, at least 100 ng/day, or at least 200 ng/day.
40. The formulation according to any one of embodiments 28-39, wherein the formulation retains physical stability, retains chemical stability, and retains from 10% to 120% of the biological activity measured at the time of initial testing of the formulation.
41. A method of preparing the formulation as defined in any one of embodiments 28-40 by contacting a biologically active ingredient as defined in any one of embodiments 1-27 with a carrier.
42. A method for maintaining or improving the healthy appearance of the skin in an individual and or the rejuvenation of the skin of an individual that comprises topical administration to an individual of a skin care composition protein as defined in any one of embodiments 1-27 or the formulation as defined in any one of embodiments 28-40 in an amount that maintains or improves the healthy appearance of the skin and/or rejuvenates the skin of an individual.
43. The method according to embodiment 40, wherein the individual is suspected of having or has been diagnosed as having diabetes, celiac disease, acne (facial actinic keratoses), inflammatory conditions, has a decrease in circulating hormones such as growth hormone or estrogen, or the individual has been exposed to ultraviolet (UV) radiation.
44. A method of maintaining or improving or rejuvenating a skin feature or function in an individual by administering the skin care composition as defined in any one of embodiments 1-27 or the formulation as defined in any one of embodiments 28-40 to an individual.
45. The method of embodiment 44, wherein the feature or function to be maintained or improved is selected from one or more of fine lines and wrinkles; age spots and dyspigmentation; decreased skin texture, tone and elasticity; roughness, photodamage; abnormal skin epidermal thickness; decreased skin thickness; decreased smoothness, tightness of skin; age spots; fine and coarse lines and wrinkles; fine and coarse periorbital wrinkles; deeper or more abundant nasolabial folds; facial fine and coarse lines; decreased skin radiance, decreased evenness of color or pigmentation; decreased skin firmness; hyperpigmentation; dark spots and/or patches; decreased skin brightness and healthy appearance; intrinsically and extrinsically aged skin; abnormal skin cellular turnover; decreased skin barrier; decreased skin hydration or ability to retain water; brown and red blotchiness; redness; reduction of dermal epidermal junction; loss of density or individual thickness of hairs; increased pore size and number of pores; or a combination thereof.
46. The method according to embodiment 44 or 45, wherein the composition improves a feature of the skin by about 1% to about 100%, about 2% to about 98%, about 5% to about 90%, about 5% to about 80%, about 5% to about 70%, about 5% to about 60%, about 5% to about 40%, about 5% to about 30%, about 5% to about 20%, about 10% to about 80%, about 10% to about 70%, about 10% to about 60%, about 10% to about 50%, about 10% to about 40%, about 20% to about 80%, about 20% to about 70%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.
47. The method according to any one of embodiments 44-46, wherein the function improved is trans-epithelial water loss.
48. The method according to any one of embodiments 44-47, wherein the feature improved is fine lines and wrinkles.
49. The method according to any one of embodiments 44-48, wherein the feature improved is redness.
50. The method according to any one of embodiments 44-49, wherein the feature improved is loss of density or individual thickness of hairs.
51. A kit comprising a skin care composition comprising two or more active ingredients, wherein, one or more active ingredients are selected from the group of growth factors, anti-oxidants, curcumins, oils and/or red rice extracts for topical application in order to maintain or improve the healthy appearance of the skin of an individual and/or rejuvenate the skin of an individual.
52. The kit according to embodiment 51, wherein the two or more active ingredients are selected from the group of growth factors, anti-oxidants, curcumins, oils and/or red rice extracts.
53. The kit according to embodiment 52, wherein the growth factor is selected from EGF, Heparin-binding EGF-like growth factor (HB-EGF), transforming growth factor-α (TGFα), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4), TGFβ1, TGFβ2, TGFβ3, inhibin-α, activin-β (forms A-C, E), anti-müllerian hormone, bone morphogenetic proteins (BMP1-11, & 15), and growth and differention factors (GDFs 1-3, 5-11) decapentaplegic, Lefty1, ESA, EPO, IGF1, IGF2, insulin and Nodal.
54. The kit according to embodiment 52, wherein the anti-oxidant is selected from Vitamin C (ascorbate), vitamin B3 (niacinamide and its derivatives), Vitamin E (α-, β-, and γ-tocopherols, tocopherol sorbate, tocopherol acetate, other esters of tocopherol; phenols, such as butylated hydroxy benzoic acids and their salts, 6-hydroxy-2,5,7,8-tetramethylchroman-2-carboxylic acid; gallic acid and its alkyl esters, especially propyl gallate; uric acid and its salts and alkyl esters; sorbic acid and its salts; lipoic acid; amines (e.g., N,N-diethylhydroxylamine, amino-guanidine); sulfhydryl compounds (e.g., glutathione), dihydroxy fumaric acid and its salts; glycine pidolate; arginine pilolate; nordihydroguaiaretic acid; bioflavonoids; curcuminoids (e.g., curcumin, tetrahydrocurcumin, desmethoxycurcumin and bis-desmethoxycurcumin and their esters); lysine; methionine; proline; superoxide dismutase; silymarin; tea extracts; grape skin/seed extracts; melanin; and rosemary extracts.
55. The kit according to any one of embodiments 51-54, wherein the active ingredient is the growth factor EGF, epoetin (EPO), IGF1, or TGFβ3.
56. The kit according to any one of embodiments 51-55, wherein the first biologically active ingredient is an ESA and the second biologically active ingredient is selected from an EGFP, an IGF, and a TGFβ.
57. The kit according to embodiment 56, where the first biologically active ingredient is an ESA and the second biologically active ingredient is an EGFP.
58. The kit according to embodiment 56, where the first is an ESA and the second biologically active ingredient is an IGF.
59. The kit according to embodiment 56, where the first biologically active ingredient is an ESA and the second biologically active ingredient is a TGFβ.
60. The kit according to embodiment 56, where the first biologically active ingredient is an EGFP and the second biologically active ingredient is an IGF.
61. The kit according to embodiment 56, where the first biologically active ingredient is an EGFP and the second biologically active ingredient is a TGFβ.
62. The kit according to embodiment 56, where the first biologically active ingredient is an IGF and the second biologically active ingredient is a TGFβ.
63. The kit according to any one of embodiments 51-62, further comprising a third biologically active ingredient.
64. The kit according to embodiment 63, wherein the first biologically active ingredient is an ESA, the second biologically active ingredient is EGFP, and the third biologically active ingredient is an IGF.
65. The kit according to embodiment 63, wherein the first biologically active ingredient is an ESA, the second biologically active ingredient is EGFP, and the third biologically active ingredient is a TGFβ.
66. The kit according to embodiment 63, wherein the first biologically active ingredient is an ESA, the second biologically active ingredient is biologically active ingredient an IGF, and the third biologically active ingredient is a TGFβ.
67. The kit according to embodiment 63, wherein the first biologically active ingredient is an EGFP, the second biologically active ingredient is an IGF, and the third biologically active ingredient is a TGFβ.
68. The kit according to any one of embodiments 51-66, further comprising a fourth biologically active ingredient.
69. The kit according to embodiment 68, wherein the first biological active ingredient is that of an EGFP, the second biologically active ingredient is an IGF, and the third biologically active ingredient is a TGFβ, and fourth biologically active ingredient is an ESA or wherein the first biological active ingredient is that of an EGF, the second biological active ingredient is that of an IGF-1, the third biological active ingredient is that of an TGFβ-3, and the fourth biological active ingredient is that of an Erythropoietin-α.
70. The kit according to any one of embodiments 51-69, further comprising an additional active ingredient selected from the group consisting of an anti-inflammatory, an anti-oxidant, and a humectant.
71. The kit according to embodiment 70, comprising a curcuminoid.
72. The kit according to embodiment 70, comprising an ascorbate.
73. The kit according to embodiment 70, comprising a curcuminoid and an ascorbate.
74. The kit according to embodiment 70, comprising a curcumin and an ascorbic acid.
75. The kit according to any one of embodiments 51-74, wherein the biological activity of the active ingredients is expressed in activity units, wherein each of the activities is present from between about 0.0001 U/ml and about 100 U/ml, between 0.001 U/ml and about 10 U/ml, between 0.01 U/ml and about 100 U/ml, between 0.1 U/ml and about 100 U/ml, between 1 U/ml and about 100 U/ml, between 0.01 U/ml and about 1000 U/ml, between 0.1 U/ml and about 500 U/ml, between 1 U/ml and about 100 U/ml.
76. The kit according to any one of embodiments 51-75, wherein the protein is present at in the range of from about 0.01 pg/ml to about 100 ng/ml, from about 0.1 pg to about 100 ng/ml, from about 1.0 pg/ml to about 400 ng/ml, from about 0.001 ng/ml to about 400 ng/ml, from about 0.01 ng/ml to about 400 ng/ml, from about 0.1 ng/ml to about 400 ng/ml, about 1.0 ng/ml to about 400 ng/ml, about 5 ng/ml to about 400 ng/ml, about 10 ng/ml to about 400 ng/ml, about 20 ng/ml to about 400 ng/ml, about 50 ng/ml to about 400 ng/ml, or about 100 ng/ml to about 1000 ng/ml.
77. The kit according to any one of embodiments 51-76, wherein the biological activity is expressed as activity units (U) and the protein is present at in the range of from about 0.01 U/ml to about 10 U/ml, about 0.01 U/ml to about 15 U/ml, about 0.01 U/ml to about 20 U/ml, about 0.01 U/ml to about 25 U/ml, about 0.01 U/ml to about 30 U/ml, about 0.01 U/ml to about 35 U/ml, about 0.01 U/ml to about 40 U/ml, about 0.01 U/ml to about 45 U/ml, about 0.01 U/ml to about 50 U/ml, about 0.01 U/ml to about 75 U/ml, or about 0.01 U/ml to about 100 U/ml. In another aspect, the composition applied to a individual provides one or more proteins in an amount at least 0.01 U/ml, at least 0.01 U/ml/day, at least 0.1 U/ml, at least 1.0 U/ml, at least 5.0 U/ml, at least 10 U/ml, at least 20 U/ml, at least 50 U/ml, at least 100 U/ml, or at least 200 U/ml.

### EXAMPLES

The following non-limiting examples are provided for illustrative purposes only in order to facilitate a more complete understanding of the disclosed subject matter. These examples should not be construed to limit any of the embodiments described in the present specification, including those pertaining to the compositions or methods or uses of treating skin.

### Example 1

### Preparation of a Cream, Gel, or Lotion or Liposomal Prep

A skin care composition for use of an individual is formulated to include the following active ingredients at the following concentration

| Active | Concentration | |
|---|---|---|
| TGF-β3 | 2 | ng/ml |
| EGF | 2 | ng/ml |
| IGF-1 | 2 | ng/ml |
| rhEPO | 1 | ng/ml |
| Curcumin | 1 | mg/ml |
| Ascorbic Acid | 30 | mg/ml |

### Example 2

### Panel Testing of the Preparation

A study will enroll a total of 60-200 Caucasian female individuals aged 40 to 65 years with moderate to moderately severe evidence of skin aging, such as periorbital wrinkles. Following a 2-week washout period in which the participants use a mild facial cleanser ad libitum and a facial moisturizer twice daily, the individuals are divided into two treatment groups. One group will use a skin care composition of the present invention formulated as a cream daily in the morning and a night cream daily in the evening, each over the entire face, while the other group use a morning application of the same emollient base without the added active ingredients.

After 8 weeks of use, a cohort of 25 individuals from each treatment group (selected before treatment is to begin) may continue treatment for an additional 16 weeks (total of 24 weeks).

At baseline and after 8 and 24 weeks, standardized high resolution digital images are taken of both the left and right sides of the patients' faces to capture the changes in facial wrinkles. These images are evaluated by expert grading for improved appearance of fine lines and wrinkles (+8 to −8 grading scale for improvement or worsening in the after treatment image) and are also assessed by computerized image analysis for changes in periorbital fine line and wrinkle area.

Skin barrier integrity on each side of each individual's face may also be determined via measurement of trans-epidermal water loss (TEWL) at the same time points. Individuals' skin is also clinically graded for erythema (redness) and dryness periodically throughout the study, including baseline, 8 and 24 weeks, as well as intermediate times, to insure a thorough assessment of tolerance to treatment.

After 8 weeks, expert visual grading and wrinkle image analysis can show that the treatment regimen is as effective at reducing the appearance of fine lines and wrinkles. The percent improvement of the appearance of wrinkles can be calculated for each individual by taking the score at 8 or 24 weeks, dividing by the score at baseline, and multiplying by 100%. Similarly, the improvement in the TEWL can be calculated by taking the measurement at week 8 or 24 weeks, dividing by the measurement at baseline, and multiplying by 100%. The improvement in the individual's grade for redness or dryness can also be calculated from the grades at week 8 or 24 relative to that at baseline.

### Example 3

### Clinical Trial of the Preparation

the objective of this efficacy trial for was to determine if the synergistic and systematic use of a cosmetic collection, comprising three distinct products, Day Serum, Day Moisturizer, and Nighttime Moisturizer (Night Renewal), would effectuate the appearance of various factors of facial skin. The Nighttime Moisturizer comprised about 40 ng/ml of EGF, about 40 ng/ml of IGF-1, about 40 ng/ml of TGF-β3, and about 40 ng/ml of Erythropoietin-α. The 60-day clinical trial enrolled 30 subjects ranging in age from 33-69 with a median age of 51 and a mean age of 49. Subjects were requested to adhere to a wash out period of 7 days where no anti-aging products were to be used. Subjects were further instructed to have no treatments to their skin for 7 days prior to the commencement of the study, including: acid peels, dermabrasion, laser treatments, botulinum toxin treatments, and dermal filler treatments.

Subjects began using the three test products on their face one day after they tested it on their forearm to check for reactions. For each day of the 60-day trial, each subject was to apply 1-3 pumps of Day Serum to entire face in the morning (6 am to 9 am), and then allow this application to dry for 5 minutes. Each subject was then to immediately apply 1-3 Pumps of Day Moisturizer to entire face, and allowed this application to set for 5 minutes. In the evening, each subject was to apply 1-3 pumps of Night Moisturizer to entire face prior to retiring for the evening. Subjects were instructed to reapply Day Serum and Day Moisturizer if they washed their face, subsequent to morning application, and prior to 5:00 PM.

Investigators made visual observations both at the commencement of the trial (Day-1, before treatment)) and at the end of the trial (Day-60, after treatment). Subjects were also photographed on Day-1 and Day-60 with high-resolution photographic equipment. Images included front facial, left side and right side views; both right and left periorbital areas, forehead, and right and left corners around the mouth and nose. Analysis by investigators as assessed both by visual observations photographic images included the following four skin factors: 1) Fine Lines & Wrinkles, 2) Plumpness, 3) Firmness, and 4) Moisture. In addition, on Day-60, an self-evaluation survey of the treatments and products was administered to the subjects, where each had the opportunity to express his/her own experiences in whether the products created the appearance of reduced fine lines and wrinkles, reduction in spots, evenness of skin tone, and increased plumpness and firmness and overall skin moisture. Twenty-two subjects, of the original thirty, returned for the follow-up evaluation. Three subjects dropped out of the trial due to concerns of subsequent medical conditions, unrelated to the product trial. Five subjects failed to appear at the Day-60 mark.

Appearance of fine line and wrinkle reduction. Comparison of Day-1 and Day-60 findings assessed by investigator observations and high-resolution photographic analysis showed that the appearance of fine lines were reduced, on average, by up to 27% in the majority of subjects, based on the use of all three products. There were photographic examples, in some subjects, where some of the finer lines appeared to be diminished by as much as 75%. The primary areas where fine lines appeared to be most diminished were in the periorbital areas, forehead, and the mid-line between the lower lip and the chin and on the broad areas of the left and right cheeks.

Appearance of increased plumpness. Comparison of Day-1 and Day-60 findings assessed by investigator observations and high-resolution photographic analysis showed that the appearance of skin plumpness to have increased by as much as 25%, based on the use of all three products. The appearance of plumpness was most notable in the periorbital areas, the left and right cheeks, and within one-inch left, right, up and down areas of the nose.

Appearance of increased firmness. Comparison of Day-1 and Day-60 findings assessed by investigator observations and high-resolution photographic analysis showed that the combined use of all three products produced an overall appearance in skin firmness by 20%. The appearance of firmness was noticeable on the entire facial area, and in particularity on sides of the face adjacent to the mouth and periorbital areas.

Moisture. Comparison of Day-1 and Day-60 findings assessed by investigator observations and high-resolution photographic analysis showed that that the combined use of all three products produced an overall appearance in increased skin moisture by as much as 16%. The highest increase in skin moisture occurred in the population of subjects who declared, prior to the trial, that they were prone to dry skin. This population showed an appearance of the increase in moisturization of 16%. Subjects that described themselves as having neither dry nor oily skin appeared to have a 7% increase in skin moisture. Subjects that described their skin as being oily appeared to have a 9% increase in skin moisture. The overall appearance of skin moisture, amongst the entire subject population appeared to show an increase of 16%, ±5%.

Taken together, the results from this clinical trial demonstrate that after 60 days of using the Day Serum, Day Moisturizer and Night Renewal there is significant improvement in the facial skin with regard to reduction in fine lines and wrinkles. In addition, the smoothness, firmness and texture of the skin also appeared visibly enhanced, looking smoother and more moisturized. These results reveal that there is significant evidence and confirmation of the efficacy of this novel and scientifically innovative skin care system.

In closing, it is to be understood that although aspects of the present specification are highlighted by referring to specific embodiments, one skilled in the art will readily appreciate that these disclosed embodiments are only illustrative of the principles of the individual matter disclosed herein. Therefore, it should be understood that the disclosed individual matter is in no way limited to a particular methodology, protocol, and/or reagent, etc., described herein. As such, various modifications or changes to or alternative configurations of the disclosed individual matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the individual matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

1. A skin care composition comprising two or more active ingredients capable of maintaining or increasing the health of the skin of an individual and/or rejuvenating the skin of an individual.

2. The composition according to claim 1, wherein the two or more active ingredients are selected from the group of growth factors, anti-oxidants, curcumins, oils and/or red rice extracts.

3. The composition according to claim 2, wherein the growth factor is selected from EGF, Heparin-binding EGF-like growth factor (HB-EGF), transforming growth factor-α (TGFα), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4), TGFβ1, TGFβ2, TGFβ3, inhibin-α, activin-β (forms A-C, E), anti-müllerian hormone, bone morphogenetic proteins (BMP1-11, & 15), and growth and differention factors (GDFs 1-3, 5-11) decapentaplegic, Lefty1, ESA, EPO, IGF1, IGF2, insulin and Nodal.

4. The composition according to claim 2, wherein the anti-oxidant is selected from Vitamin C (ascorbate), vitamin B3 (niacinamide and its derivatives), Vitamin E (α-, β-, and γ-tocopherols, tocopherol sorbate, tocopherol acetate, other esters of tocopherol; phenols, such as butylated hydroxy benzoic acids and their salts, 6-hydroxy-2,5,7,8-tetramethylchroman-2-carboxylic acid; gallic acid and its alkyl esters, especially propyl gallate; uric acid and its salts and alkyl esters; sorbic acid and its salts; lipoic acid; amines (e.g., N,N-diethylhydroxylamine, amino-guanidine); sulfhydryl compounds (e.g., glutathione), dihydroxy fumaric acid and its salts; glycine pidolate; arginine pilolate; nordihydroguaiaretic acid; bioflavonoids; a curcuminoid; lysine; methionine; proline; superoxide dismutase; silymarin; tea extracts; grape skin/seed extracts; melanin; and rosemary extracts.

5. The composition according to claim 1, further comprising a third biologically active ingredient.

6. The composition according to claim 5, wherein the first biologically active ingredient is an ESA, the second biologically active ingredient is EGFP, and the third biologically active ingredient is an IGF.

7. The composition according to claim 5, wherein the first biologically active ingredient is an ESA, the second biologically active ingredient is EGFP, and the third biologically active ingredient is a TGFβ.

8. The composition according to claim 5, wherein the first biologically active ingredient is an ESA, the second biologically active ingredient is biologically active ingredient an IGF, and the third biologically active ingredient is a TGFβ.

9. The composition according to claim 5, wherein the first biologically active ingredient is an EGFP, the second biologically active ingredient is an IGF, and the third biologically active ingredient is a TGFβ.

10. The composition according to claim 1, further comprising a fourth biologically active ingredient.

11. The composition according to claim 10, wherein the first biological activity is that of an EGFP, the second biologically active ingredient is an IGF, and the third biologically active ingredient is a TGFβ, and fourth biologically active ingredient is an ESA.

12. The composition according to claim 1, further comprising an additional active ingredient selected from the group consisting of an anti-inflammatory and a humectant.

13. The composition according to claim 1, wherein the biological activity of the active ingredients is expressed in activity units, and wherein each of the activities is present from between about 0.0001 U/ml and about 100 U/ml, between 0.001 U/ml and about 10 U/ml, between 0.01 U/ml and about 100 U/ml, between 0.1 U/ml and about 100 U/ml, between 1 U/ml and about 100 U/ml, between 0.01 U/ml and about 1000 U/ml, between 0.1 U/ml and about 500 U/ml, between 1 U/ml and about 100 U/ml.

14. The composition according to claim 1, wherein the biological activity of the active ingredients is expressed in activity units, and wherein each of the activities is present in an amount of at least 0.01 U/ml, at least 0.01 U/ml/day, at least 0.1 U/ml, at least 1.0 U/ml, at least 5.0 U/ml, at least 10 U/ml, at least 20 U/ml, at least 50 U/ml, at least 100 U/ml, or at least 200 U/ml.

15. A formulation comprising the composition as defined in claim 1, and a pharmaceutically or cosmetically acceptable carrier.

16. The formulation according to claim 15, wherein the formulation further comprises one or more of a moisturizing agent or humectant, a pH adjusting agent, a deodorant or odor absorbing agent, a fragrance, a chelating agent, an emulsifying agent, a thickener, a solubilizing agent, a penetration enhancer, a colorant, a UV absorbent, an anti-oxidant agent, and a surfactant.

17. A method for maintaining or improving the healthy appearance of the skin in an individual and or the rejuvenation of the skin of an individual, the method comprises the step topical administration of a composition as defined in claim 1 in an amount that maintains or improves the healthy appearance of the skin and/or rejuvenates the skin of the individual.

18. A method according to claim 17, wherein the individual is suspected of having or has been diagnosed as having diabetes, celiac disease, acne, inflammatory conditions, has a decrease in circulating hormones such as growth hormone or estrogen, or the individual has been exposed to ultraviolet (UV) radiation.

19. A method of maintaining or improving or rejuvenating a skin feature or function in an individual by administering the skin care composition as defined in claim 1 to an individual.

20. A kit comprising a skin care composition as defined in claim 1 for topical application in order to maintain or improve the healthy appearance of the skin of an individual and/or rejuvenate the skin of an individual.
